(19) United States
(12) Patent Application Publication
(10) Pub. No.: US 2026/0031188 A1
(43) Pub. Date: Jan. 29, 2026

(54) SYSTEMS AND METHODS FOR CLASSIFYING CELLS

(71) Applicant: CEDARS-SINAI MEDICAL CENTER, Los Angeles, CA (US)

(72) Inventors: Joseph LOWNIK, Los Angeles, CA (US); Sumire KITAHARA, Manhattan Beach, CA (US); Akil MERCHANT, Los Angeles, CA (US); Tucker LEMOS, Los Angeles, CA (US)

(73) Assignee: CEDARS-SINAI MEDICAL CENTER, Los Angeles, CA (US)

(21) Appl. No.: 19/334,788

(22) Filed: Sep. 19, 2025

Related U.S. Application Data
(63) Continuation-in-part of application No. PCT/US2024/020536, filed on Mar. 19, 2024.
(60) Provisional application No. 63/491,261, filed on Mar. 20, 2023.
(60) Provisional application No. 63/506,306, filed on Jun. 5, 2023.
(60) Provisional application No. 63/590,126, filed on Oct. 13, 2023.
(60) Provisional application No. 63/596,898, filed on Nov. 7, 2023.
(60) Provisional application No. 63/830,355, filed on Jun. 25, 2025.

(51) Int. Cl. G16B 40/10

(52) U.S. Cl. CPC G16B 40/10

(57) ABSTRACT

A method for classifying one or more cells comprises receiving data associated with the one or cells, the data including, for each respective cell, information associated with one or more measurable parameters of the respective cell; inputting at least a portion of the data into a machine learning model; and receiving, from the machine learning model, an indication of an identity of at least one of the one or more cells.

### CROSS-REFERENCE TO RELATED APPLICATIONS

This application claims priority to and the benefit of U.S. Provisional Patent Application No. 63/830,355, filed Jun. 25, 2025, and is a continuation-in-part of International Application No. PCT/US2024/020536, filed Mar. 19, 2024, which claims priority to and the benefit of U.S. Provisional Patent Application No. 63/596,898, filed Nov. 7, 2023, U.S. Provisional Patent Application No. 63/590,126, filed Oct. 13, 2023, U.S. Provisional Patent Application No. 63/506,306, filed Jun. 5, 2023, and U.S. Provisional Patent Application No. 63/491,261, filed Mar. 20, 2023, each of which is hereby incorporated by reference herein in its entirety

### TECHNICAL FIELD

The present disclosure relates generally to systems and methods for sorting cells, and more particularly, to machine learning models trained to classify cells into a plurality of different phenotypes based on flow cytometry data.

### BACKGROUND

The implementation of flow cytometry into clinical diagnostics has revolutionized the field of hematopathology and has improved diagnostic capabilities tremendously. However, interpretation of clinical flow cytometry data is inherently difficult due to complex and heterogenous immunophenotypes observed in multidimensional space. Additionally, classic flow cytometry analysis is fundamentally subjective when incorporating manual gating strategies for the identification of cell populations. While significant advances have been made in both technologies for flow cytometry acquisition as well as flow cytometry data analysis, these advances have been slow to make their way into clinical diagnostics. Thus, new systems and methods for classifying cells based on flow cytometry data (and/or other types of data) are needed.

### SUMMARY

According to some implementations of the present disclosure, a method for classifying one or more cells comprises receiving data associated with the one or cells, the data including, for each respective cell, information associated with one or more measurable parameters of the respective cell; inputting at least a portion of the data into a machine learning model; and receiving, from the machine learning model, an indication of an identity of at least one of the one or more cells.

The above summary is not intended to represent each implementation or every aspect of the present disclosure. Additional features and benefits of the present disclosure are apparent from the detailed description and figures set forth below.

### BRIEF DESCRIPTION OF THE FIGURES

The foregoing and other advantages of the present disclosure will become apparent upon reading the following detailed description and upon reference to the drawings.

FIG. 1 shows a flowchart of a method for classifying cells, according to aspects of the present disclosure.

FIG. 2A is a dimensionality reduction plot showing cell classes in a sample of cells, according to aspects of the present disclosure.

FIG. 2B is a dimensionality reduction plot showing cells with a CD19 molecule in the sample of cells, according to aspects of the present disclosure.

FIG. 2C is a dimensionality reduction plot showing cells with a CD45 molecule in the sample of cells, according to aspects of the present disclosure.

FIG. 3A is a dimensionality reduction plot showing classes of cells in the sample at a “Class” level, according to aspects of the present disclosure.

FIG. 3B is a dimensionality reduction plot showing classes of cells in the sample at a “Primary” level, according to aspects of the present disclosure.

FIG. 3C is a dimensionality reduction plot showing classes of cells in the sample at a “Secondary” level, according to aspects of the present disclosure.

FIG. 3D is a dimensionality reduction plot showing classes of cells in the sample at a “Tertiary” level, according to aspects of the present disclosure.

FIG. 3A is a dimensionality reduction plot showing classes of cells in the sample at a “Indication” level, according to aspects of the present disclosure.

FIG. 4 is a flowchart illustrating the workflow of a decision support system to evaluate the monotypic population in a sample of cells, according to aspects of the present disclosure.

FIG. 5A is a maturation plot showing the presence of CD34 molecules and CD117 molecules in a sample of myeloid cells, according to aspects of the present disclosure.

FIG. 5B is a maturation plot showing the presence of CD13 molecules and CD15 molecules in a sample of myeloid cells, according to aspects of the present disclosure.

FIG. 5C is a maturation plot showing the presence of CD64 molecules and CD14 molecules in a sample of myeloid cells, according to aspects of the present disclosure.

FIG. 6 is a collection of pseudotemporal development plots showing the development of myeloid cell samples and monocyte samples over time compared to control samples, according to aspects of the present disclosure.

FIG. 7 shows a system for implementing a method for classifying cells, according to aspects of the present disclosure.

FIG. 8 shows a flowchart of a method for generating an abnormality score for each of a plurality of cell groups, according to aspects of the present disclosure.

While the present disclosure is susceptible to various modifications and alternative forms, specific implementations and embodiments have been shown by way of example in the drawings and will be described in detail herein. It should be understood, however, that the present disclosure is not intended to be limited to the particular forms disclosed. Rather, the present disclosure is to cover all modifications, equivalents, and alternatives falling within the spirit and scope of the present disclosure as defined by the appended claims.

### DETAILED DESCRIPTION

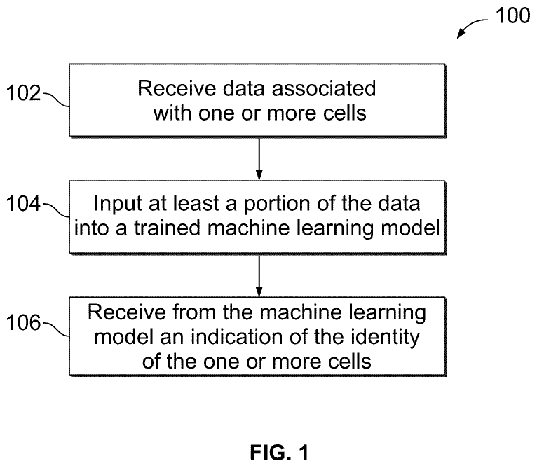

FIG. 1 shows a flowchart of a method 100 for classifying cells using machine learning methods, according to aspects of the present disclosure. Step 102 of method 100 includes receiving data associated with one or more cells. The data can include, for each respective cell, information associated with one or more measurable parameters of the respective cell. In some implementations, the data is flow cytometry data, and includes information associated with the forward-scatter (e.g., forward-scatter height and/or forward-scatter area) and side-scatter (e.g., side-scatter height and/or side-scatter area) of the respective cell, information associated with whether the respective cell contains specific biomarkers or molecules (such as various different cluster of differentiation (CD) molecule). Thus, the parameters can include parameters associated with the scattering of light (e.g., forward-scatter amount, forward-scatter height, forward-scatter area, forward-scatter angle, forward-scatter time-of-flight, side-scatter amount, side-scatter height, side-scatter area, side-scatter angle, side-scatter time-of-flight, etc.), parameters associated with a biomarker of the cell (e.g., whether or not a specific biomarker (e.g., a CD molecule) is present in the cell, an amount of the specific biomarker, an intensity of fluorescent emission from the cell, a color of fluorescent emission from the cell, etc.), and other parameters.

Any suitable molecule may be used as a biomarkers, such as a CD molecule, an antigen, an antibody, an immunoglobulin chain, or any combination thereof. The CD molecule can be a CD2 molecule, a CD3 molecule, a CD4 molecule, a CD5 molecule, a CD7 molecule, a CD8 molecule, a CD10 molecule, a CD11 molecule, a CD13 molecule, a CD14 molecule, a CD16 molecule, a CD19 molecule, a CD20 molecule, a CD22 molecule, a CD23 molecule, a CD33 molecule, a CD34 molecule, a CD45 molecule, a CD64 molecule, a CD117 molecule, an HLA-DR molecule, or any combination thereof. The immunoglobulin chain can be a kappa (κ) light chain, a lambda (λ) light chain, a gamma (γ) heavy chain, a delta (δ) heavy chain, an alpha (α) heavy chain, a mu (μ) heavy chain, an epsilon (ϵ) heavy chain, or any combination thereof.

Step 104 of method 100 includes inputting at least a portion of the data into a machine learning model, which can include any suitable machine learning model, including those discussed herein. Step 106 includes receiving from the machine learning model an indication of the identity of one or more of the cells. The indication of the identity of the cells can include placement of each cell into one or more cell classes and/or cell sub-classes (which may also be referred to as a cell cluster and a cell metacluster). For each respective class, a given cell can be determined to belong to and/or correspond with one of the subclasses of that respective class, which results in the placement of the given cell within the respective class. In general, different classes can have different levels of classification (e.g., some classes may be more “zoomed-in” or “zoomed-out” than other classes). Each class will generally correspond to a plurality of potential combinations of one or more characteristics, and each subclass within a respective class will generally correspond to a distinct one of the plurality of potential characteristics of that respective class. In some implementations, each subclass itself may be formed from a plurality of sub-subclasses, and this division can continue further as needed. A given class and/or subclass can generally be any descriptive annotation that describes a cell based on any number of different characteristics/properties (such as size, granularity, the presence/diminished presence/absence of one or more cell surface markers, the presence/diminished presence/absence of one or more intra cell markers, etc.). In general, method 200 can include sorting the cells into any number of various different cell classes and/or cell sub-classes, where each class and/or sub-class is associated with a distinct combination of characteristics and/or placement in specific subclasses of one or more classes, and this placement may be based on the one or more measurable parameters of the cells.

In an example, one class is a phenograph class, and the plurality of subclasses include a plurality of distinct predefined phenographs. In an additional example, one class is referred to as cluster class, and the plurality of subclasses include a plurality of distinct classes.

In another example, one class is an immunophenotype class, which include various different subclasses associated with distinct combinations of the presence, absence, and diminished presence of various different molecules, such as CD molecules. In some of these examples, each subclass is associated with a distinct combination of (i) a presence of one or more CD molecules, one or more cell surface markers, one or more intracellular markers, or any combination thereof; (ii) a diminished presence of the one or more CD molecules, the one or more cell surface markers, the one or more intracellular markers, or any combination thereof; (iii) an absence of the one or more CD molecules, the one or more cell surface markers, the one or more intracellular markers, or any combination thereof; or (iv) any combination of (i)-(iii).

The immunophenotype class could include any combination of the following subclasses: (i) a first subclass associated with the presence of a CD10 molecule; (ii) a second subclass associated with the presence of a CD5 molecule, the diminished presence of a CD20 molecule, the diminished presence of a CD22 molecule, and the absence of a CD23 molecule; (iii) a third subclass associated with the presence of the CD5 molecule, the presence of the CD20 molecule, the presence of the CD22 molecule, and the presence of the CD23 molecule; (iv) a fourth subclass associated with the presence of the CD5 molecule, the diminished presence of the CD20 molecule, the diminished presence of the CD22 molecule, and the presence of the CD23 molecule; (v) a fifth subclass associated with the diminished presence of CD5 molecule, the diminished presence of the CD20 molecule, the diminished presence of the CD22 molecule, and the absence of the CD23 molecule; (vi) a sixth subclass associated with the diminished presence with the CD5 molecule, the diminished presence of the CD20 molecule, the diminished presence of the CD22 molecule, and the diminished presence of the CD23 molecule; (vii) a seventh subclass associated with the absence of the CD5 molecule, the presence of the CD20 molecule, the diminished presence of the CD22 molecule, and the absence of the CD23 molecule; (viii) an eighth subclass associated with the absence of the CD5 molecule, the presence of the CD20 molecule, the diminished presence of the CD22 molecule, and the diminished presence of the CD23 molecule; (ix) a ninth subclass associated with the absence of the CD5 molecule, the presence of the CD20 molecule, the diminished presence of the CD22 molecule, and the diminished presence of the CD23 molecule; and (x) a tenth subclass associated with the absence of the CD5 molecule, the presence of the CD20 molecule, the presence of the CD22 molecule, and the absence of the CD23 molecule.

In an additional example, the plurality of classes includes a CD5/CD10 class, and wherein the plurality of subclasses of the CD5/CD10 class includes a first subclass associated with a presence of a CD10 molecule, a second subclass associated with a presence of a CD5 molecule, a third subclass associated with a diminished presence of the CD5 molecule, and a fourth subclass associated with an absence of the CD5 molecule. In a further example, one class can include a B-cell normality class, where the subclasses include a subclass of normal B-cells and a subclass of abnormal B-cells.

Thus, in various examples, the indication of the identity of each respective cell can include an indication of whether the respective cell is a B-cell, an indication of whether the respective cell is a normal B-cell or an abnormal B-cell, an indication of a predefined phenotype of a plurality of predefined phenotypes that the respective cell belongs to, an indication of an immunophenotype of the respective cell, and any combinations thereof.

In a further example, the indication of the identity of each respective cell includes a placement of each respective cell into one of a plurality of cell classes that include B-cells, normal B-cells, abnormal B-cells, B-cells having any combination of a presence or an absence of one or more cluster of differentiation (CD) molecules, T-cells, normal T-cells, abnormal T-cells, T-cells having any combination of a presence or an absence of one or more cluster of differentiation (CD) molecules, double-negative T-cells, cells negative for a CD45 molecule, granulocytes, monocytes, monocytes with a diminished presence of a CD4 molecule, monocytes with a presence of a CD56 molecule, mature cells, immature cells, natural killer (NK) cells, NK cells with an absence of a CD2 molecule and a CD5 molecule, NK cells with an absence of the CD5 molecule, plasma cells, B-lymphoblasts, T-lymphoblasts, or any combination thereof.

In some implementations, each cell can be placed into multiple classes. For example, there may be two different sets of a plurality of classes, where the classes of the first set are associated with distinct combinations of a first group of characteristics, and the classes of the second set are associated with distinct combinations of a second group of characteristics. The machine learning algorithm can place each cell into one of the classes of the first set and one of the classes of the second set. The first and second sets could each include the same number of classes, or could include different numbers of classes.

The classes and sub-classes can be defined based on sorting algorithms such as FlowSOM, Phenotype, etc. In some implementations, sub-classes are the most granular level on which the cells can be classified (e.g., there is no more distinct categorization than a sub-class), while classes are generally larger levels of classification (e.g., a single class can contain multiple sub-classes, etc.). However, in general, the machine learning model can be configured to analyze data associated with a population of one or more cells, and generate some indication of the identity of the cells. The training data can include raw flow cytometry associated with a plurality of cells, and a determination of the identity of each respective cell in the plurality of cells. The raw flow cytometry data, can include, for each cell, data (e.g., fluorescence data) associated with the presence/diminished presence/absence of a cell marker (e.g., cell surface marker, intracell marker, etc.) in the cell, data (e.g., forward scatter data) associated with the size of the cell, data (e.g., side scatter data) associated with the granularity of the cell, etc. Generally, data associated with any type of light-scattering related property of the cell and/or any type of flow cytometry data can be used to train the machine learning algorithm. The training data can be generated from predetermined sorting algorithms, manual gating/annotation (e.g., manual sorting/identification of the cells), etc. In general, any annotation of a discrete population of cells been sorted into a large number of different classes and/or subclasses can be used to train the machine learning model.

In some implementations, the machine learning model is a random forest model with a plurality of trees and a voting module. Each of the trees is configured to generate an independent indication of the identity of each respective cell and/or placement of each respective cell into one or more of the classes. The voting module can select the output of one of the trees, or could determine a weighted average of at least two of the trees.

In some implementations, the machine learning model is a k-nearest neighbor model where k=7. In some implementations, the machine learning model includes any combination of a neural network, a k-nearest neighbor algorithm, a decision tree, and a random forest model.

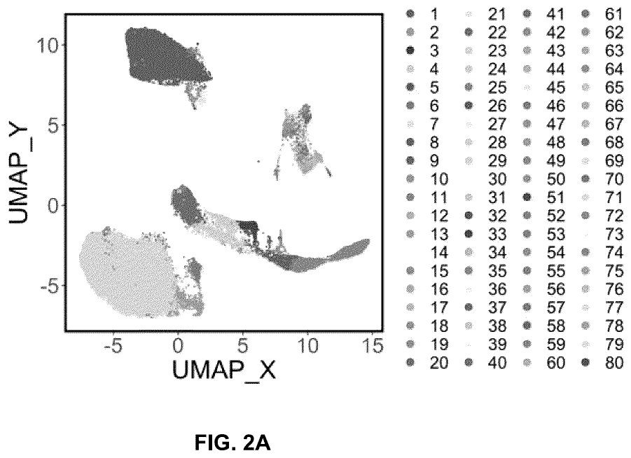

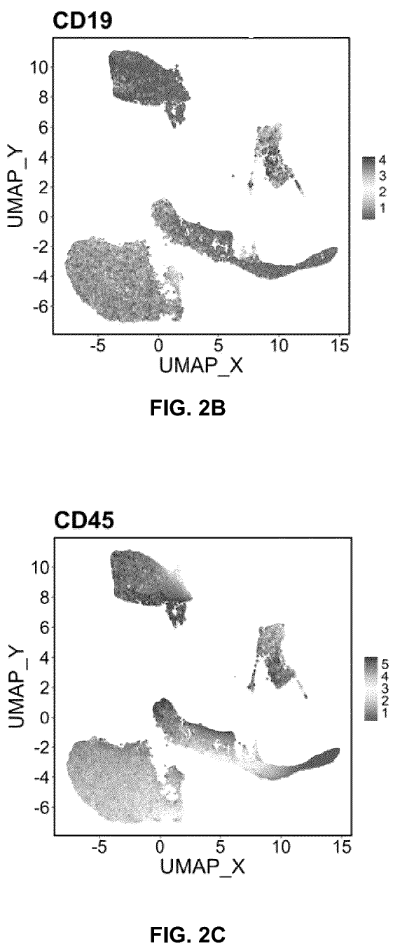

FIGS. 2A-2D illustrate examples of the placement of cells into different classes and subclasses, showing how the systems and methods disclosed herein are able to effectively identify classes and sub-classes within a sample of cells. FIG. 2A shows an example dimensionality reduction plot highlighting individual cell sub-classes in a sample of B-cells that can be identified using the disclosed features. In the illustrated implementations, UMAP dimensionality reduction was used to reduce the number of cell characteristics/properties into a UMAP_X variable and a UMAP_Y variable, in order to plot all of the different sub-classes. As shown, 80 different sub-classes were identified. FIGS. 2B and 2C show example dimensionality reduction plots highlighting different antigen expression levels in the sample of B-cells. FIG. 2B shows a plot highlighting cells with a CD19 molecule, and FIG. 2C shows a plot highlighting cells with a CD34 molecule. FIG. 2D shows a heatmap of all the sub-classes identified within the sample of B-cells.

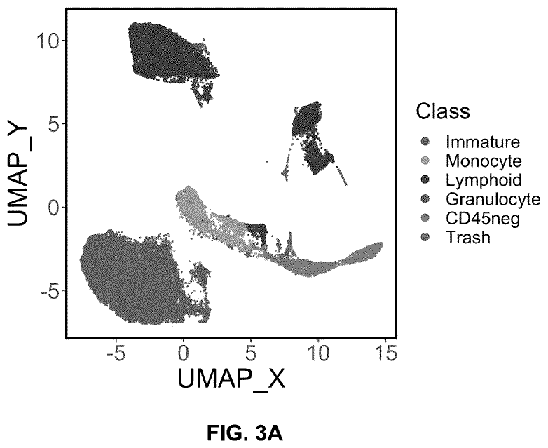

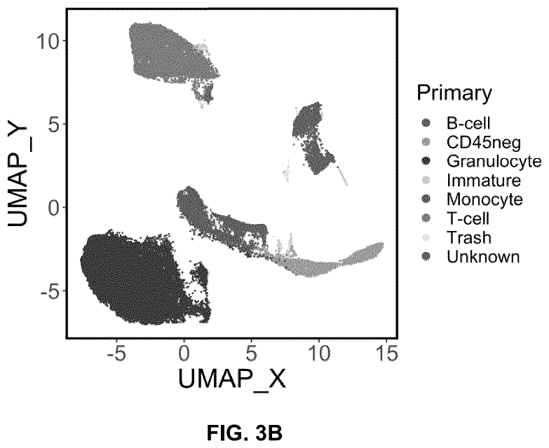

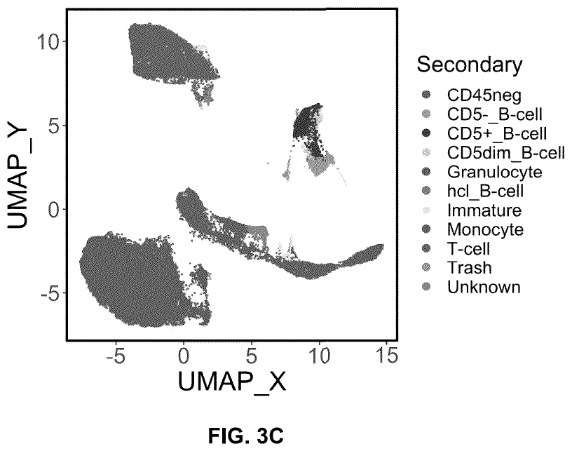

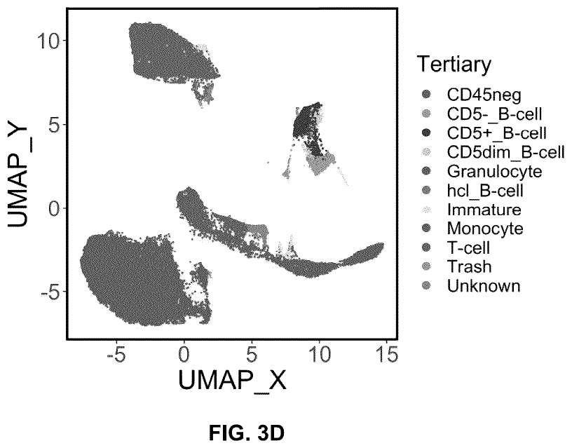

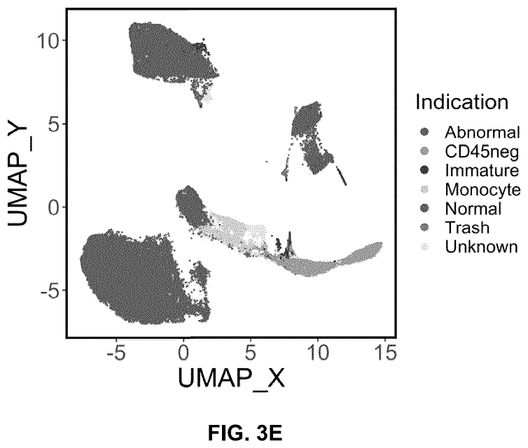

In some implementations, the dimensionality reduction plots can be annotated with different characteristics of the cells to provide clinically relevant information. These annotations can be done at different levels to provide varying degrees of information. FIGS. 3A-3E illustrate such annotations. FIG. 3A is a dimensionality reduction plot showing annotation of cells at the “Class” level, where the cells are divided into immature cells, monocytes, lymphoids, granulocytes, CD45 negative cells, and a trash/discard group.

FIG. 3B is a dimensionality reduction plot showing annotation of cells at the “Primary” level, where the cells are divided into B-cells, CD4 positive T-cells, CD34 negative cells, granulocytes, immature cells, immature T-cells, immature T-NK cells, monocytes, NK-cells, plasma cells, T-cells, a trash/discard group, and unknown cells.

FIG. 3C is a dimensionality reduction plot showing annotation of cells at the “Secondary” level, where the cells are divided into B-cells, CD4 positive T-cells, CD45 negative cells, CD5 positive B-cells, CD5 positive NK-cells, CD7 negative NK-cells, CD8 positive T-cells, granulocytes, immature cells, immature T-cells, immature T-NK cells, monocytes, NK-cells, double negative (DN) T-cells, double positive (DP) T-cells, granulocytes, human T-lymphotropic virus (HTLV) CD4 positive T-cells, immature cells, immature T-cells, immature T-NK cells, monocytes, NK cells, plasma cells, a trash/discard group, and unknown cells.

FIG. 3D is a dimensionality reduction plot showing annotation of cells at the “Tertiary” level, where the cells are divided into B-cells; CD2 positive granulocytes; CD38 positive B-cells; T-cells that are CD4 positive and CD3 negative; T-cells that are CD4 positive, CD56 positive, and have a diminished presence of CD7; T-cells that are CD4 positive and CD7 negative; CD4 positive T-cells; CD45 negative cells; CD5 positive B-cells; B-cells that are CD5 positive and CD38 positive; B-cells that are CD5 positive and CD7 positive; CD5 positive NK-cells, CD56 positive monocytes; T-cells that are CD7 negative, CD8 positive, and have a diminished presence of CD2; T-cells that are CD7 negative and CD8 positive; CD7 negative NK-cells; T-cells that are CD8 positive and CD56 positive; CD8 positive large granular lymphocytes (LGL); CD8 positive T-cells; DN T-cells; DP T-cells; DP T-cells that are CD7 negative; granulocytes; HTLV CD4 positive T-cells; immature cells; immature T-cells; immature T-NK-cells; monocytes; NK-cells; plasma cells; a trash/discard group; and unknown cells.

FIG. 3E is a dimensionality reduction plot showing annotation of cells at the “Indication” level, where the cells are divided into abnormal cells, CD45 negative cells, immature cells, normal cells, a trash/discard group, and unknown cells.

In some implementations, the output of the machine learning model can be one or more self-organizing map (SOMs) with adaptive sizing, where the grid size adapts to the number of cells in each class/subclass, etc. This preserves the resolution of the classes/subclasses across low- and high-count specimens.

This data can be input into a decision support system that is configured to analyze the data and generate a variety of different types of information that can be used by a healthcare provider to make decisions for the patient from which the cells originated from. In some implementations, the decision support system is configured to determine cell population frequencies, which can be the frequency of a given cell class and/or cell subclass within the entire cell population. In some implementations, the decision support system is configured to generate a graphical representation of the cell population that includes indications of the various different classes and/or subclasses into which the cells have been sorted. In some implementations, the decision support system is configured to generate recommendations for further testing based on the different classes and/or subclasses into which the cells have been sorted, the cell population frequencies, etc. In some implementations, the decision support system is configured to generate recommended text for reporting various features of the cell population. In some implementations, the decision support system is configured to generate a differential diagnosis of the patient based on the cell population frequencies, and/or other aspects of the cell population.

In some implementations, the decision support system utilizes a rules-based algorithm to determine its output. In some of these implementations, if a given cell population includes certain cell classes/sub-classes, and or certain cell classes/sub-classes with certain frequencies, the decision support system can provide a specific type of output. For example, if the ratio of the number of cells in a CD4 class/subclass to the number of cells in a CD8 class/subclass is greater than a threshold (such as 7), than the decision support system can designate the cell population as having an “Increased CD4:CD8” status. The decision support system may generate various types of outputs and/or take various type of actions that are associated with that status. In another example, if a threshold percentage of T-cells (such as 8%) are positive for both the CD4 molecule and the CD8 molecule, then the decision support system can designate the cell population as having an “Increase dual positive T-cells” status, and generate outputs/take actions based on that status. A non-limiting list of example status includes: Increased CD4:CD8; Decreased CD4:CD8; Subtle CD4 changes; HTLV CD4; Pan T-cell subtle changes; Elevated CD4:CD8 with subtle antigenic changes; Increased dual positive T-cells; Increased gamma delta T-cells; LGL; Monocytosis; Monocytosis and CD56; Aberrant CD56 without monocytosis; Left-shifted myeloid; Absolute eosinophilia; Prominent eosinophilia; and Absolute basophilia.

The decision support system can provide a variety of different types of outputs. In some implementations, the decision support system provides a template for reporting the status of the cell population. In some implementations, the decision support system provides information on how to interpret the different class/subclass frequencies of the cell population. In some implementations, the decision support system provides recommended next steps for a healthcare provider (which could include a patient's physician and/or another worker such as a nurse, laboratory technician, etc.), such as further analysis of the cell population (using the techniques disclosed herein and/or other conventional techniques), other tests to order and/or perform, suggested patient history follow-up, etc.

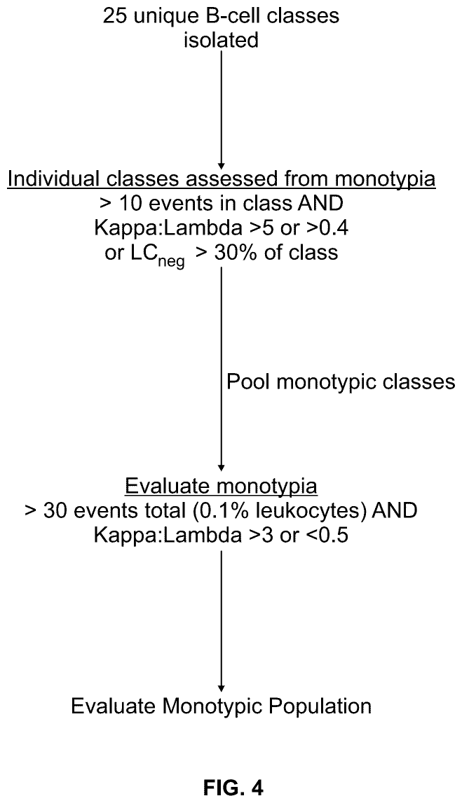

FIG. 4 is a flowchart that illustrates the workflow of a decision support system in analyzing B-cells to diagnose different forms of cancer. First, the cell population is analyzed to identify different B-cell classes. In the illustrated implementation, 25 classes are identified, but any suitable number may be used. Next, the identified classes are assessed to identify monotypic classes in a polytypic background. In this implementation, a monotypic cell class is one that includes at least 10 events (e.g., individual identified cells) with a kappa (κ) light chain:lambda (2) light chain ratio greater than 5 or less than 0.4, or where greater than 30% of the cells in the class are light chain negative (LC_{neg}). Next, the monotypic classes are pooled and evaluated again to determine the total monotypic population. In this step, the leukocyte percentage and the kappa:lambda ratios are evaluated in the classes initially labeled as monotypic. Classes with at least 0.1% leukocytes (generally at least 30 events) and a kappa:lambda ratio of greater than 3 or less than 0.5 are placed into the final monotypic population, which can then be evaluated.

In the example of FIG. 4, the monotypic population can be evaluated based on the presence of six markers: CD22 molecules, CD20 molecules, kappa light chains, lambda light chains, CD23 molecules, and CD5 molecules. Different preliminary diagnoses can be generated based on these markers, and certain follow-up tests recommended. For example, a first set of thresholds for the markers results in a preliminary diagnosis of chronic lymphocytic leukemia (CLL), with instructions to order a fluorescence in situ hybridization (FISH) test, a tumor protein 53 gene (TP53) test, a next-generation sequencing (NGS) test, and a somatic hypermutation test.

A second set of thresholds for the markers results in a preliminary diagnosis of CLL, acute CLL (aCLL), or mantle cell lymphoma (MCL), with instructions to order a t(11;14) FISH test, a TP53 NGS test, and a somatic hypermutation test. A third set of thresholds for the markers results in a preliminary diagnosis of lymphoplasmacytic lymphoma (LPL) or marginal zone lymphoma (MZL), with instructions to order an MYD88 sequencing test with CXCR4 reflex, a serum immunoglobulins test, a serum protein electrophoresis (SPEP) test, and an immunofixation (IFE) test. A fourth set of thresholds for the markers results in a preliminary diagnosis of hairy cell leukemia (HCL), with instructions to order a BRAF V600E sequencing test.

The decision support system can also be used to evaluate cell populations in any other desired fashion. In some implementations, the decision support system is used to identify T-cell abnormalities, such as CD8 positive T-cell large granular lymphocytic leukemia (T-LGL). For example, T-LGL can be analyzed based on the presence of CD2 molecules, CD3 molecules, CD4 molecules, CD7 molecules, CD8 molecules, DP T-cells, and DN T-cells. In some implementations, the decision support system is used to evaluate myeloid cells for the presence of monocytes, granulocytes, blasts, eosinophils, and basophils.

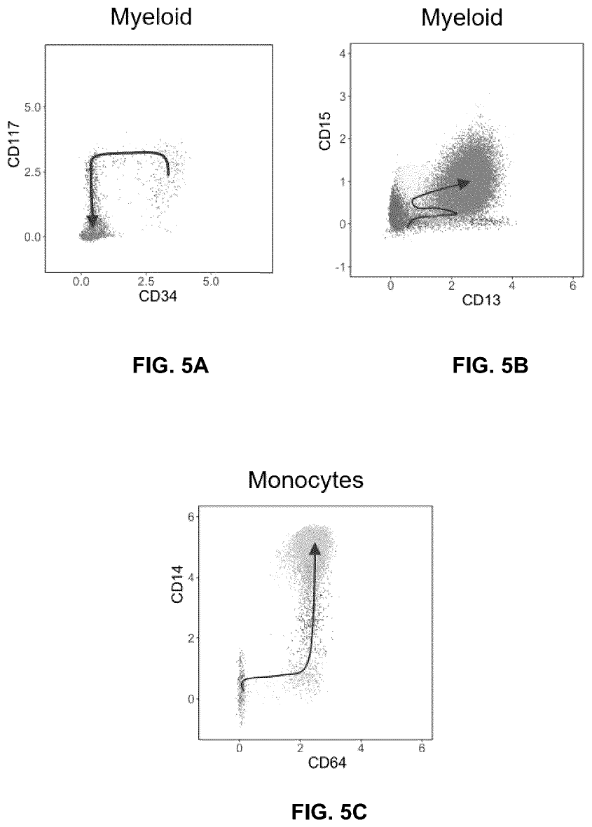

Referring now to FIGS. 5A-5C, in some implementations, maturation patterns of different cell classes and/or subclasses can be analyzed and shown (for example as part of the decision support system). A first example is shown in FIG. 5A, which is a maturation plot shows the presence of CD34 molecules and CD117 molecules in myeloid cells within a sample. The amount of CD34 molecules is plotted on the horizontal axis, and the amount of CD117 molecules is plotted on the vertical axis. Different subclasses of myeloid cells (e.g., myeloid cells with different maturities) will generally have different amounts of these two markers, and thus by determining the amount of these markers in the myeloid cells of the sample, the maturation of the myeloid cells in the sample can be tracked and visualized.

A second example is shown in FIG. 5B, which shows the presence of CD13 molecules and CD15 molecules in myeloid cells within a sample. The amount of CD13 molecules is plotted on the horizontal axis, and the amount of CD15 molecules is plotted on the vertical axis. Again, different subclasses of myeloid cells (e.g., myeloid cells with different maturities) will generally have different amounts of these two markers, and by determining the amount of these markers in the myeloid cells of the sample, the maturation of the myeloid cells in the sample can be tracked and visualized.

A third example is shown in FIG. 5C, which shows the presence of CD64 molecules and CD14 molecules in monocytes within a sample. The amount of CD13 molecules is plotted on the horizontal axis, and the amount of CD14 molecules is plotted on the vertical axis. Again, different subclasses of monocytes (e.g., monocytes with different maturities) will generally have different amounts of these two markers, and by determining the amount of these markers in the monocytes of the sample, the maturation of the monocytes in the sample can be tracked and visualized.

In general, the value of any two or more parameters within any class or subclass of cells can be determined to show the differentiation between the further subclasses of the cells. This analysis can be done for generally any type of cell, such as myeloids, monocytes, lymphoids, and others. Further, the two parameters can be the presence of generally any biomarker, including all possible antigenic markers that can be used to define cell states and/or cell types, and/or other parameters. Thus, in some implementations, step 106 of method 100 includes receiving an indication of the identity of a plurality of cells, wherein the indication of the identity includes the value of a first parameter associated with a first biomarker of the cells, and the value of a second parameter associated with a second biomarker of the cells. Method 100 can further include identifying a plurality of distinct maturation stages of the plurality of cells based on the values of the first and second parameters for each cell. In some cases, the plurality of cells are myeloid cells, the first biomarker is a CD34 molecule, and the second biomarker is a CD117 molecule. In other cases, the plurality of cells are myeloid cells, the first biomarker is a CD13 molecule, and the second biomarker is a CD15 molecule. In further cases, the plurality of cells are monocytes, the first biomarker is a CD64 molecule, and the second biomarker is a CD14 molecule.

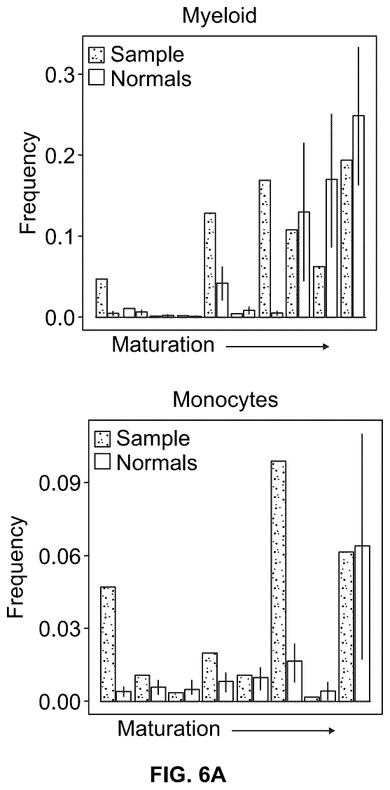

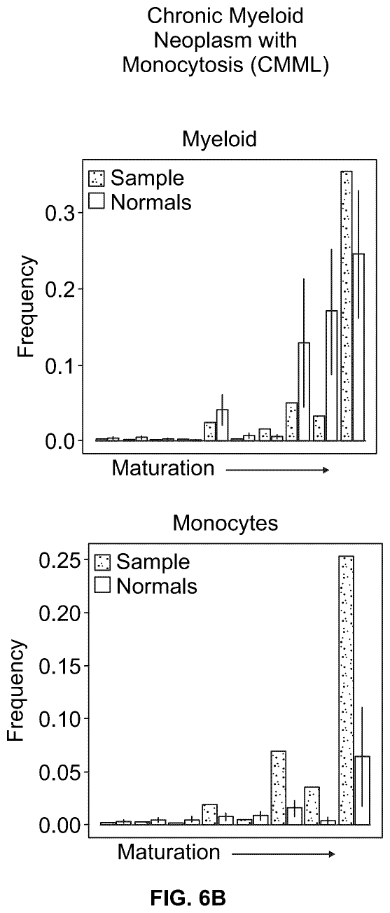

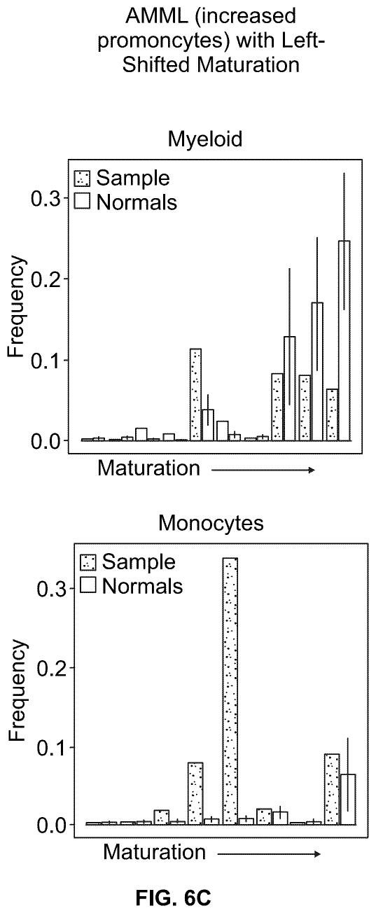

In some implementations, pseudotemporal development can be applied to cell classes to show different maturation stages of the cells of the sample, and compare them to control samples. Pseudotemporal development refers to the maturation stages of a sample of cells that would occur over time, but are visualized in a static setting. For example, FIG. 6 shows pseudotemporal development plots of myeloid cells and monocytes that compare the maturation of these cells to control samples. As illustrated, pseudotemporal development can be used to show increased blasts with left-shifted maturation (indicative of acute myelomonocytic leukemia, or AMML) in myeloid cells (myeloblasts) and monocytes (monoblasts); chronic myeloid neoplasm with monocytosis (chronic myelomonocytic leukemia or CMML); and AMML (increased promonocytes) with left-shifted maturation, for example. The control samples can be any sample suitable for the test being performed, such as a normal sample (e.g., a sample known to not have any cancerous cells, cell variations, cell abnormalities, etc.), a sample showing a specific disease state or regenerative state (e.g., a sample collected during post-chemotherapeutic marrow regeneration), and others.

Other statistical information can also be generated and/or displayed. For example, the expression range of various different biomarkers can be plotted for multiple different classes/subclasses, and compared to normal cells. Bimodal expression can also be tested for and displayed. In another implementation, a heatmap of different cell classes/subclasses can be shown. In these heatmaps, different stages of the maturation of a larger cell class (e.g., myeloids) can be shown in each column, and where the column for each maturation stage includes separate indicators of the expression of multiple biomarkers. These heatmaps can be used to show detailed information about abnormalities in various different stages of cell maturation.

In some implementations, various aspects of the present disclosure can be used to perform MRD testing (also referred to as Minimal Residual Disease testing or Measurable Residual Disease testing), for example for acute myeloid leukemia (AML), acute myelomonocytic leukemia (AMML), B-cell acute lymphoblastic leukemia (B-ALL), T-cell acute lymphoblastic leukemia (T-ALL), other lymphoblastic leukemias, plasma cell neoplasms, mature B-cell neoplasms (such as chronic lymphocytic leukemia (CLL)), and others. The MRD testing can be used to detect remaining cancer cells, which can guide future treatment options for a patient. AML cells tend to be very heterogeneous and undergo both molecular and immunophenotypic changes post-treatment. By analyzing cell samples using the techniques described herein, core reference populations in the cell samples can easily be recognized and analyzed.

Described herein is an example technique for population identification in a cell sample. First, flow cytometry events (e.g., cell detections) due to fluidic issued can be removed by analyzing fluorophore values versus time. Next, assessment of forward scattering height (FSC-H) vs. forward scattering area (FSC-A) can be used to remove doublets, and live/dead staining can be used to remove dead cells. Erythoid cells can then be identified by comparing CD45 molecules vs CD71 molecules. Non-erythroid cells can then be roughly gated for leukocytes, granulocytes, immature cells, and monocytes by analyzing side scatter height vs. CD45 molecules. Leukocytes can then be removed based on CD13 vs. CD33 negativity. Next, monocytes can be gated based on CD64 positivity and CD14 vs. HLA-DR expression. Plasmacytoid dendritic cells and basophils can be identified based on CD123 vs. HLA-DR expression, and immature cells can be identified based on CD34 vs. CD117 expression.

Different treatments (e.g., different chemotherapy regiments) can have different effects on the immunophenotypes of the cancer cells. The cancer cells can also undergo CHIP-associated mutations, which can increase the difficult of traditional MRD testing. The machine learning models and methods discussed herein can be used to annotate all cell populations based on developmental trajectories. Having granular refinement in the classes/subclasses allows for statistical testing of aberration of specific immunophenotypes, as well as aberrations in developmental trajectories of cell lineage. Thus, more accurate testing for and diagnosing of various hematologic malignancies and/or disease states can be performed, including more accurate MRD testing.

The decision support system can provide the information in any suitable format. For example, in some implementations, the decision support system can show a diagnosis, an interpretation of the cell population with relevant cell measurements (e.g., white blood cell count, hemoglobin count, etc.), graphs showing various different cell classes/subclasses, information about the patient's clinical history, etc. The decision support system may also include a number of options that can be controlled by the user (who may be the patient's healthcare provider, a laboratory technician, etc.). These options can include which graphs to show, which classes/subclasses to show on the graphs, how the graphs are formed, how the information is presented, etc.

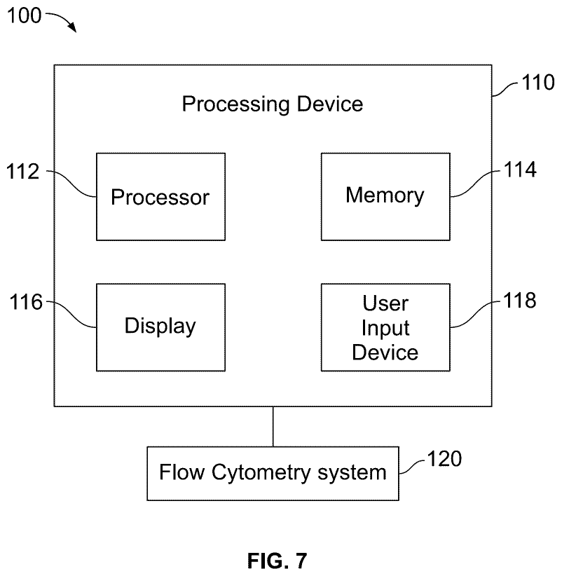

FIG. 7 is a block diagram of an example system 100 for implementing any of the herein-discussed features and processes. For example, system 100 can be used to implement a machine learning model that classifies cells as discussed herein. The system 100 can include one or more processing devices 100, which can each include any one or more of a processor 112, a memory 114, a display 116, a user input device 118, and/or other components. The memory 114 can include machine-readable instructions for executing one or more machine learning models. The processor 112 can execute these instructions to implement the one or more machine learning models. The memory 114 can also store data associated with the cells that are being analyzed (e.g., flow cytometry data).

The processing device 110 can include any suitable processing device, such as general purpose computer systems, microprocessors, digital signal processors, micro-controllers, application specific integrated circuits (ASICs), programmable logic devices (PLDs) field programmable logic devices (FPLDs), programmable gate arrays (PGAs), field programmable gate arrays (FPGAs), mobile devices such as mobile telephones, personal digital assistants (PDAs), or tablet computers, local servers, remote servers, wearable computers, or the like. The memory device 114 can include any suitable memory device and/or machine-readable medium that is capable of storing, encoding, and/or carrying a set of instructions for execution by a processing device and that cause the processing device to perform and/or implement any of the features discussed herein, including solid-state memories, optical media, magnetic media, random access memory (RAM), read only memory (ROM), a floppy disk, a hard disk, a CD ROM, a DVD ROM, flash memory, or other computer readable medium that is read from and/or written to by a magnetic, optical, or other reading and/or writing system that is coupled to the processing device, can be used for the memory or memories.

The display 116 can be used to display any information associated with the features disclosed herein, including the results of the classification analysis by the machine learning model. The display device 116 can be any known display technology, including but not limited to display devices using Liquid Crystal Display (LCD) or Light Emitting Diode (LED) technology. The user input device 118 can be used to allow the user to interact with the system 100 for any suitable purpose, including initiating, pausing, or terminating the analysis by the machine learning model; adjusting any parameters of the analysis, etc. In some implementations, the system 100 includes a flow cytometry system 120 that generates the data. The flow cytometry system 120 can generally be any suitable type of flow cytometry system. In other implementations, the system 100 does not include the flow cytometry system, but instead receives data from an external source.

Discussed below is a first example associated with the various features disclosed herein. Aspects of this example can be implemented using the system 100 of FIG. 7.

### Materials and Methods

Patient samples and data set collection: Flow cytometry data was obtained from 2,300 routine diagnostic peripheral blood diagnostic samples from patients between Jan. 1, 2020 and Dec. 30, 2021 at Cedars-Sinai Medical Center. These samples included a wide range of hematologic disorders as well as normal samples. The flow cytometry panel consisted of three 10-color tubes and a tube for viability testing. Samples with a viability <75% were excluded from the analysis. A target of 30,000 events per tube were acquired using a Navios cytometer (Beckman Coulter, Miami, FL). Flow cytometry was originally collected as an LMD files and further converted to an FCS 3.0 file using FCS Express. No initial gating was performed prior to downstream analysis.

Clustering and dimensionality reduction: R statistical software was used for all clustering and machine learning applications. FCS files were initially processed and transformed using an arcsine transformation with a cofactor of 5 using the Spectre cytometry package. The ˜63e6 cells for each tube were randomly downsampled to 5e6 cells and clustered using FlowSOM with 20×20 clusters and 80 metaclusters per tube using all fluorescent as well as forward scatter (FSC) and side scatter (SSC) for tubes 1 and 3. For the B-cell tube, clustering was done using all fluorescent parameters as well as FSC and SSC but did not include surface light chains. UMAP dimensionality reduction plots were generated using the same clustering parameters as was used for FlowSOM clustering.

Cluster annotation: FlowSOM metaclusters were annotated by a group of expert hematopathologists based on immunophenotypes and scatter characteristics. Each cluster was given a detailed annotation with subsequent less granular annotations. Populations with a high staining background suggestive of non-specific staining were removed from the analysis given a designation of trash to be removed from subsequent analysis. Populations with abnormal immunophenotypes were examined by mapping these clusters back to patients for a more detailed understanding of the abnormal population.

Machine learning & modeling: The caret package in R was utilized for all machine learning training. Tubes 1-3 were individual trained to predict FlowSOM metaclusters using the same fluorescent and scatter parameters used for initial FlowSOM clustering and UMAP dimensionality reduction. Random Forest was conducted using 1,000 trees and mtry was initially scanned for accuracy with an mtry of 3 being used for final modeling. For KNN, a k=7 was used after initial screening of k from 1 to 50. Neural net was set with a tune grid of 50.

Prediction and Reporting: R Markdown was used for all report generation for patient samples. Briefly, all 4 collected .fcs files for a patient were imported using the Spectre package. Doublets were removed based on the forward scatter time of flight parameter (FSC TOF). Each individual cell from Tubes 1-3 was assigned a FlowSOM metacluster using the Random Forest model. Annotations for each individual cell were applied based on FlowSOM metacluster for that cell. Surface light chain expression was arithmetically assigned based on expression of Kappa vs. Lambda light chain. Clinical interpretation scripts were applied based on specific annotation frequencies for each tube.

General Model Design: While several groups have recently reported the utilization of machine learning for clinical flow cytometry diagnoses, most of these studies focused on analyzing sample files as a global picture for a diagnosis. A different approach was chosen that utilizes newly described clustering methods to cluster all cells into immunophenotypically unique clusters and subsequently trained machine learning models to classify cells into these clusters (FIG. 3). This automated cell identification was then incorporated into an automated annotation and analysis platform which scripted a clinical interpretation for each sample based on identified cells (FIG. 3). For this study, 2,300 patient samples from 2020-2021 were utilized. Each sample had 4 assays completed, consisting of a T/NK-cell assay, B-cell assay, myeloid/monocytic assay as well as a viability assay.

High Dimensional Clustering and Population Identification: Sample files from each assay (except the viability assay) were concatenated and randomly down sampled from ˜63×10⁶ events per assay to 10e⁶ events per assay. FlowSOM was used for population clustering. A 20×20 SOM with 80 metaclusters per tube (not including viability tube) was used. A high metacluster number was chosen to overestimate the number of immunophenotypically unique clusters instead of a lower metacluster number which could miss subtle immunophenotypic differences between cell populations that may be of clinical significance. All collected parameters were used for FlowSOM metaclustering for the T/NK-cell assay and myeloid/monocytic assay. All collected parameters except for surface immunoglobulin light chain (Kappa and Lambda) were utilized for clustering of the B-cell tube. Expression levels for analyzed antigens for each tube was used for initial FlowSOM metacluster annotation: NK-cell tube; B-cell tube; myeloid/monocytic tube.

Each FlowSOM metacluster was annotated as a distinct cell population with a few non-specific and/or uninterpretable metaclusters per assay, which were excluded from subsequent analysis. As there were many metaclusters which were immunophenotypically similar, multiple levels of classification with increasing granularity were developed, as well as an annotation as to whether the population had a normal or abnormal immunophenotype. Annotations for rare metaclusters which represented <0.1% of all sampled events were confirmed by manual examination of samples with the highest frequencies of that specific metacluster. Relationships of annotation levels to antigen expression were examined using UMAP dimensionality reduction (FIGS. 2-4).

### Random Forests Outperform Other Machine Learning Algorithms for Predicting FlowSOM Metaclusters

FlowSOM clustering has limitations for use in clinical flow cytometry analysis because if each new sample is run through the FlowSOM algorithm independently, it is likely that different clusters would emerge between samples and each cluster would have to be reannotated. To address this problem, it was examined whether machine learning algorithms would be able to accurately predict FlowSOM metaclusters using a large training set with >2,300 samples. From the FlowSOM clustering sets for each assay, the 5e6 cells/assay were randomly down-sampled to 3,000 cells per FlowSOM metacluster for each assay for model training. Of the training models tested, random forest and K-nearest neighbors (knn) had the highest accuracy. However, the random forest model predictions were notably faster than the knn predictions. Being that the FlowSOM metaclusters are more granular than is clinically relevant, model performance for the multiple levels of annotation (Class, Primary, Secondary, Tertiary) was examined for each FlowSOM metacluster. Not surprisingly, progressing to less granular annotations (CD4+ T-cell vs. CD8+ T-cell, etc.) resulted in higher accuracy for all models tested.

### T/NK-Cell Decision Support System Development

Automated interpretation for the T/NK cell tube focused heavily on the most common T/NK aberrations seen in clinical flow cytometry. Initial CD4:CD8 ratio is calculated and reported. If any immature (blast) population which expresses a T-cell antigen is detected, it is automatically flagged for review. Established were tiered thresholds for T-cell antigenic abnormalities based on the likelihood that the given antigenic abnormality represents a clonal population (FIG. 2A). For both CD4 and CD8 T-cell populations, Tier 1 consists of the loss of CD3 or CD2, Tier 2 consists of the loss of CD5, and Tier 3 consists of the loss of CD7 (FIG. 2A). Additionally, inverted CD8 ratios can be added to the tiered systems to adjust the thresholds. Thresholds can be set for other T-cell populations such as CD4+CD8+ double-positive T-cells as well as double negative T-cells. Relevant plots and annotations are generated automatically to describe abnormal cell populations such as T-LGL populations (FIGS. 2B-D). As annotations are classified as likely normal or possible abnormal, this annotation level can also be plotted for ease of interpretation (FIG. 2E).

### B-Cell Decision Support System Development

While B-cells can have immunophenotypic irregularities which may suggest abnormality, light chain restriction is the ultimate standard of a B-cell population being flagged as atypical. As use surface light chain expression were not used in the initial B-cell clustering and prediction model, populations were not partitioned on the basis of light chain expression. There were 25 metaclusters which represented B-cells in the B-cell tube. For each individual metacluster, monotypia was calculated as a kappa:lambda ratio of >5 or <0.4 with a minimum of 10 events necessary for flagging. All monotypic clusters are then compiled and reexamined for monotypia with a minimum event frequency of 0.1% of leukocytes (FIG. 3A). If a monotypic population is identified, the immunophenotype is determined and a differential diagnosis and suggested ancillary studies are recommended (FIG. 3A). Additionally, FSC characteristics can be automatically analyzed for the given monotypic population to assess for large cell/prolymphocytic transformation.

### Myeloid/Monocytic Decision Support System Development

In addition to assessing granulocytes and monocytes, the myeloid/monocytic tube in the assay is useful for flagging immature populations. The high dimensional clustering allows for accurate assessment of left-shifted myeloid cells, which can be flagged at a specified percentage (FIG. 4A). Basophilia, eosinophilia and monocytosis can be calculated based population frequencies from clustering and an inputted WBC. Abnormal CD56 on monocytes is assessed using the T/NK tube results as the specific marker is not represented in the myeloid tube. Blasts/immature cells are flagged at thresholds based on the specificity of the immaturity markers they express such as CD34 and CD117 (FIG. 4A).

### Clinical Validation and Implementation

As the majority of diagnoses on peripheral blood in hematopathology aided by flow cytometry consist of low-grade B-cell neoplasms, the diagnoses of these entities was used as a benchmark for performance of the decision support system. The performance of the automated population assignment and decision support system was assessed using 1,500 retrospective cases and 500 prospective cases. With a set threshold of 0.1% of leukocytes for monotypic b-cell detection, an ROC curve demonstrated an AUC of 0.96 (FIG. 5A). Sensitivity of 100% was reached at 0.5% of leukocytes with 83% specificity. However, as missing diagnoses was not acceptable, the threshold of 0.1% of leukocytes was kept. The majority of cases which were overcalled by the decision support system as being monotypic had normal CD19, CD20, CD22, CD5 and CD23 expression (FIGS. 5B-5E) and had a predominance of being flagged as kappa-restricted (FIG. 5F). Interestingly, CD5-LPDs were more commonly overcalled, whereas populations with a CLL immunophenotype were less frequently overcalled as monotypic (FIG. 5G).

### Discussion

The integration of flow cytometry into clinical diagnostics, particularly for hematopathology, ushered in a new era of precision in cellular analysis. While this technique is invaluable, its inherent complexity, stemming from the multidimensional nature of the data and intricate immunophenotypes of various disease entities, has made data interpretation a challenging task. This study aims to simplify this process through the application of machine learning techniques, with a particular emphasis on the implementation of a decision support system to aid pathologists in the assessment and diagnosis of entities by flow cytometric analysis.

Our study set out to examine whether machine learning could be used as a wayfinding tool to both decrease analysis time as well as aid the hematopathologist in determining a diagnosis. The method differed from previous studies incorporating machine learning for clinical flow cytometry diagnostics by approaching classification at the cellular level rather than a global sample picture. The model eliminates the necessity of manual gating for population identification, thus significantly decreasing time associated with analysis both at the technician and hematopathologist level. In addition, the model is able to detect subtle immunophenotypic changes and rare populations which may otherwise be missed by manual gating strategies.

This study expands the utility of clustering methods such as FlowSOM, which traditionally have been of limited value in the clinical setting. With most clustering and dimensionality reduction algorithms being stochastic, the addition of new samples usually results in differential clustering outcomes requiring new cluster annotations for each use. Utilizing machine learning to predict clustering results from an established training set which has already been analyzed, onto new data allows for the implementation of these high-dimensional clustering algorithms in the clinical setting.

By assigning individual cells annotations based on metacluster predictions, population frequencies and summary statistics are able to be used to develop diagnostic interpretations and guide diagnoses. This program was developed such that thresholds for flagging populations for review can be set by individuals with minimal coding changes allow for increased overall customization specific to each lab. One notable limitation is that initial clustering, model training, and annotation is panel and lab specific. However, these steps only need to be completed once per panel and can be rapidly implemented using the annotations as a benchmark. Overall, it is demonstrated that the implementation of machine learning for cell type identification in flow cytometry data can act as a wayfinding tool to aid hematopathologists with diagnoses and significantly decrease the time and expertise required to analyze such data. However, it's important to note that while the methodology streamlines and adds precision to the flow cytometry data interpretation process, it doesn't eliminate the need for expert intervention. The role of the hematopathologist remains crucial, especially in situations where abnormal immunophenotypes emerge.

Discussed below is a second example associated with the various features disclosed herein. Aspects of the example can be implemented using the system 100 of FIG. 7. In general, aspects of the present disclosure associated with analyzing cell populations using machine learning to place cells into different cell groups (e.g., classes, subclasses, sub-subclasses, clusters, metaclusters, etc.) can be used to generate the data utilized in this example.

### Introduction

The importance of measurable residual disease (MRD) testing in acute leukemia is one of the most important predicting factors for patient outcomes. Initial evaluation for residual disease was done using morphological assessment, and had a sensitivity of 5%, of 1 in 20 cells and was very subjective. However, with the advancement of recent technologies such as flow cytometry and molecular techniques, the sensitivity for MRD testing has been greatly improved with abilities to distinguish leukemic from normal cells at a sensitivity down to 0.001%.

The sensitivity of flow cytometry for MRD testing is dependent on many factors, including instrumentation and analysis methods, with sensitivities ranging from 1% to 0.01%. Flow cytometry based MRD analysis is even further complicated by the heterogenous immunophenotypes seen in acute leukemia, particularly in acute myeloid leukemia (AML). Additionally, the AML immunophenotypes are known to change following chemotherapy and the normal healthy leukocyte component can show regenerative changes, further complicating the distinction between leukemic and normal marrow components. Due to these factors, not only is expert training a necessity for the analysis of AML MRD data, but a large number of analyzed antigens is required, sometimes up to 20 different antigens.

While clinical flow cytometry analysis started with a single fluorescent channel, it quickly expanded to 4 color, 8 color, and now up to 12 color panels. Current methods for detecting residual AML cells from background normal cells include the leukemia associated immunophenotype (LAIP) or different from normal (DfN) approach. While the LAIP method relies on abnormalities in the diagnostic immunophenotype of the AML, the DfN approach is applicable to all samples and examines variations in abnormalities in maturation patters allowing for recognition of residual. Regardless of the approach, flow cytometry based MRD analysis requires >20 antigenic markers, which necessitates the need for multiple assays to be run in parallel on a single sample. This necessity imposes several limitations: 1) the sample must be split into multiple tubes, thus decreasing the number of cells available per tube for acquisition, ultimately limiting sensitivity; 2) several backbone markers (CD34, HLA-DR, CD33, etc.) are needed for comparing tubes, allowing for only a few unique antigens to be added per tube and also requiring cross tube analysis relying heavily on inference, thus decreasing sensitivity and specificity.

Recent advances in flow cytometry instrumentation have expanded the number of fluorophores that can be simultaneously analyzed to >40. These methods include spectral based instruments as well as mass cytometry. Hypothesized was that utilizing spectral flow cytometry for AML MRD would increase sensitivity by allowing a for a single tube per patient, thus decreasing the inference required between tubes as well as increasing the number of cells available for analysis.

In this study, the development and evaluation of a 22-color AML MRD panel (MRD-22) on a 3-laser spectral flow cytometer is presented. Demonstrated is concordance with gold-standard clinical flow cytometry-based AML MRD testing as well as detection of low-level leukemic cells which were not detected by this gold-standard method but were detected by the method and molecular techniques. Also highlighted is the improved sensitivity for a wide variety of immunophenotypes using the MRD-22 assay as well as a standard 10-color panel. Overall, the findings showcase the feasibility of spectral flow cytometry for AML MRD testing.

### Methods

### Antibody Titration

Antibodies were titrated by starting with 2× the recommended volume for each and antibody and diluted 2-fold for a total of 8 dilutions. Titrations were tested on pooled BM aspirate samples from multiple patients. Up to 5 different antibodies were tested in a single assay for titration studies. For each titration, staining index (SI) was calculated using

$$S I = \frac{{MFI}_{pos} - {MFI}_{neg}}{2 \times S {D}_{neg}} .$$

Titrations which had the highest SI were utilized for the panel.

### Sample Handling, Staining and Flow Cytometry

BM cells were stored in EDTA tubes at 4 C° until processing. All samples were processed within 96 hours from the time of collection. Up to 3 mL of BM aspirate was lysed in a 50 mL tube using 1×RBC Lysis Buffer (BioLegend) for 10 minutes at room temperature. Cells were pelleted at 500 rcf for 5 minutes and washed with 10 mL of PBS. Live/dead staining was then performed using Zombie Aqua (BioLegend) at a 1:3000 dilution for 10 minutes at room temperature in the dark. Staining was quenched with an equal volume of Cell Staining Buffer (BioLegend). Cells were washed and incubated with TrueStain Fc block (BioLegend) for 10 minutes at room temperature followed by antibody staining in the presence of Monocyte Blocker (BioLegend) and Brilliant Violet Buffer (BD) for 30 minutes at room temperature in the dark. Total volume for staining was 150 uL. Cells were washed once with PBS and fixed with Cell Fixation Buffer (BioLegend) for 15-30 minutes followed by washing with PBS. Cells were then acquired on a Cytek Northern Lights within 3 days. Samples were run with a maximum event rate of 30,000 events/second. A target of 10 million events to be acquired per sample was set.

### Manual Flow Cytometry Data Analysis

Unmixed flow cytometry FCS files were exported and analyzed to identify general cell populations. To compare sensitivity of MRD-22 vs. conventional 10-color panels, samples were analyzed as previously stated for MRD-22 and files were split into 3 separate files with corresponding 10-color panels. For analysis of the utility of an incorporated live/dead stain, samples were analyzed as in MRD-22 but the gate for live cells was expanded to include dead cells and dead cells were only removed using scatter characteristics.

### Statistical Analysis

Limit of the blank (LOB) was calculated as LOB=mean_{blank}+3(SD_{blank}). While LOB is classically calculated as LOB=mean_{blank}+1.645(SD_{blank}), a more conservative calculation was chosen. Limit of detection (LOD) was calculated as OD=LOB+3(SD_{blank}). LOB and LOD were calculated using samples from 5 patients undergoing staging marrows for lymphomas or solid malignancies as well as 5 post-treatment marrows from AML patients which were MRD negative to allow for regenerative changes to be calculated into the LOB and LOD. As AML is heterogenous, LOB and LODs were calculated for multiple immunophenotypes of AML detected in MRD samples from this study. LOBs and LODs were calculated using both MRD-22 and the standard split M1, M2, and M3 tube assay. LOBs and LODs between MRD-22 and split assays were compared using a One-way ANOVA for each immunophenotype analyzed.

### Clustering and Dimensionality Reduction

10 normal BM samples were concatenated and downsampled to 1e6 cells followed by clustering using Phenograph (k=45, all fluorescent and scatter channels used) as well as dimensionality reduction with UMAP (all fluorescent and scatter channels used). Immunophenotypes of individual clusters were assessed and annotated based on lineage. For myeloid and monocytic lineages, stages of developmental trajectories were annotated based on established antigenic marker expression in myelomonocytic maturation. For comparison of diagnostic samples to normal BM samples, the frequencies of each cluster in 10 normal BM samples were determined and used to compare to the frequency of each cluster for the diagnostic sample.

### Results

### Panel Design and Rationale

The backbone of the panel was built to incorporate all the markers from published and well-established AML MRD panels. A few additional antigenic markers were added to tease apart cellular subsets (CD2 and CD10) as well as aid in identification of myelomonocytic maturation (CD11b and CD10). The panel also incorporated a fixable live/dead stain to aid in the removal of dead cells from analysis. The panel was designed for minimal fluorophore spillover between co-expressed antigens and the antibodies were titrated for optimal staining. This panel design allowed for easy recognition of core reference populations to aid in MRD analysis. Additionally, as all antigenic markers are analyzed in a single tube in the MRD-22 assay. Erythroid, myeloid, and monocytic maturation patterns can be thoroughly characterized simultaneously.

### MRD-22 Increases Sensitivity for Multiple Immunophenotypes

The sensitivity of flow cytometry-based AML MRD assays is dependent on the immunophenotypes of the leukemic cells in question. Leukemic cells with multiple immunophenotypic abnormalities or distinct LAIPs allow for a higher sensitivity than subtle immunophenotypic abnormalities. To examine the effects of MRD-22 on the sensitivity for the detection of leukemic cells with differing immunophenotypic abnormalities, cells were stained with an MRD-22 panel and following acquisition, the FCS file was split into multiple files such that each file would correspond to the classical M1, M2 and M3 used for AML MRD detection. For individual cases, the MRD-22 dataset, and the split (M1, M2, M3) data set were analyzed separately. The background, LOB, and LOD were examined using an AML case with abnormal CD5 and CD7 expression on blasts. The background frequencies of cells found in the leukemic gate when using the traditional MRD-10 method, MRD-10 with live/dead staining, and MRD-22 were calculated. Not surprisingly, having two abnormally expressed antigens (CD5 and CD7) led to a decreased false positive signal in the leukemic gate compared to a single antigenic abnormality (CD5).

It is recognized that dead and dying cells non-specifically bind antibody as well as exhibit autofluorescence. These properties of dead cells can drastically confound AML MRD analysis as these cells may non-specifically mimic leukemic cells. Dead cells are classically removed from analysis using scatter characteristics, exhibiting decreasing FSC and increasing SSC as cells die. Additionally, cell viability can be assessed using a myriad of different stains such as 7-AAD, Syto16, Propidium Iodide and more. However, these viability stains are usually assessed in a separate tube as to not decrease the number of antigenic markers which can be analyzed at time. The incorporation of live/dead staining decreased the background cell frequency in the leukemic gate for both the examination of abnormal CD5 alone as well as CD5/CD7 combined. MRD-22 had a ˜400 fold decrease in background cells in the leukemic gates for both CD5 alone and CD5/CD7 combined. As AML MRD assay sensitivity is dependent on the LOD and LOB of the assay, the LOD and LOB of multiple AML immunophenotypes was calculated using staging marrows as well as regenerating marrow samples. MRD-22 had a lower LOD and LOB for all leukemic immunophenotypes assessed compared to the split (M1, M2, M3) data set.

### MRD-22 Helps Clarify Confounding Cell Populations

There are several cell populations that can be troublesome for AML MRD analysis as they can be confused as leukemic cells if not analyzed carefully. These include basophils, plasmacytoid dendritic cells (pDCs), immature NK cells, as well as erythroid progenitors. In traditional panels, these populations can easily be distinguished in one or two tubes but may be difficult to discern in the remaining assay tubes, ultimately leading to inaccurate interpretation. As MRD-22 incorporates antigenic markers for discriminating multiple cell types in a single tube, these populations can easily be analyzed for all antigenic markers simultaneously. BM pDCs can be mistaken for leukemic cells if sufficient markers such as CD123 are missing from a tube due to their low CD45 expression, often overlapping blasts. Early stages of pDC maturation can have dim CD34 expression. Additionally, activated and maturing pDCs can express antigens such as CD2, CD5, CD7, or CD56 which are classically considered LAIPs. Because MRD-22 incorporates these LAIP expression markers in the same tube as CD123, pDCs are able to be distinguished from potential leukemic cells.

### MRD-22 Performs is Non-Inferior to the Gold Standard Method

AML MRD flow cytometry assays are considered the gold standard for assessment of MRD. All patient samples (n=24) for the study were sent to a well-established reference lab for AML MRD evaluation. Several patients also had molecular studies conducted on the same clinical sample. For all samples which tested positive at the outside reference laboratory, MRD-22 was in 100% agreement with these samples. Additionally, there were 3 samples which were resulted as MRD negative by the outside reference lab which were found to be MRD positive using MRD-22 and confirmed positive for NPM1 by qRT-PCR or NGS testing. An example of bm23 can be seen in FIG. 4 highlighting abnormal dim CD33, dim CD38 and absent CD117 expression on abnormal blasts.

### MRD-22 Allows for Detailed Assessment of Myelomonocytic Maturational Stages

Because most MRD assays have multiple tubes per assay, high-dimensional clustering for maturation analysis is difficult to interpret. Using Phenograph clustering for discrete population identification from the MRD-22 data, clusters representing distinct myelomonocytic maturational stages were found. Clonal myeloid neoplasms characteristically consist of altered maturational status, whether it be maturational halting at the blast stage for acute leukemias, or overall increased myelopoiesis in chronic disorders such as chronic myeloid leukemia (CML) or chronic myelomonocytic leukemia (CMML). Using normal BM samples to establish a reference range for different maturation stages as defined by unique clusters in the dataset, it was found that when compared to a new diagnostic sample, these maturational trajectory abnormalities were able to be easily visualized for CMML, acute myelomonocytic leukemia (AMML) presenting with increased blasts and left-shifted myelomonocytic maturation as well as AMML presenting with increased promonocytes rather than blasts.

### Discussion

While the ability to detect MRD to low levels (0.01%-0.1) has greatly improved the ability to clinically risk-stratify and alter treatment strategies for MRD-positive patients, a large number (25-30%) of MRD-negative patients still relapse. These relapse cases most likely represent false negative results which may be due to a number of reasons, including inadequate sensitivity. In pediatric ALL, a routine cutoff of 0.01% is used for MRD-positivity, and patients with MRD-positivity fare significantly worse than MRD-negative patients. However, patients with very low (0.001%-0.01%) level MRD positivity fare significantly worse than patients with <0.001% MRD positivity further suggesting the need for improving assay sensitivity

While molecular studies such as PCR have a higher sensitivity than most flow cytometry-based AML MRD assays, the utility of these PCR assays is limited to a small percentage of AML due to the heterogenous nature of AML as well as confounding mutations representing clonal hematopoiesis of indeterminate potential (CHIP). Additionally, flow cytometry is cheaper and has a faster turnaround time than most molecular based assays. Due to these characteristics, flow cytometry remains the mainstay for AML MRD detection.

Improving AML MRD flow cytometry assay sensitivity requires addressing both pre-analytical and analytical components of the assay. Pre-analytical methods for improving sensitivity include increasing the number of cellular events acquired per patient, using a high-quality sample, as well as strategic panel design. AML MRD analysis typically is done using either the LAIP or DfN approaches. As LAIP requires a diagnostic sample, DfN is more widely applicable, but requires significant training and relies on inference between multiple tubes to thoroughly characterize samples. The use of spectral cytometry for the MRD-22 assay addressed many of the pre-analytical and analytic components which limit assay sensitivity. By incorporating all markers into a single tube, the number of acquired cellular events can be increased as the sample does not have to be split into multiple tubes, which is particularly important for paucicellular specimens. Additionally, the collection of all panel parameters in a single assay removes the necessity to infer antigen expression on populations of interest between tubes allowing for more confident evaluation of potential MRD populations.

In this study, a spectral flow cytometry-based assay for the detection of AML MRD (MRD-22) was developed and evaluated. Due to its high parameter nature, MRD-22 is able to very thoroughly characterize myeloid, monocytic, and erythroid maturation patterns, which aid in MRD detection by the DfN approach. As this assay also incorporates multiple LAIP markers, LAIPs can be assessed on clearly defined cell populations using the large myelomonocytic maturation backbone of the panel. The addition of a live/dead stain in the same tube also significantly improves the sensitivity of the assay by removing potential non-specific staining elements thus increasing the confidence in calling low-level abnormal populations. The MRD-22 performed as well, if not better than the gold standard AML MRD flow cytometry assay. Low-level MRD samples not detected by the gold standard method were detected using MRD-22 and confirmed by molecular methods. Overall, the feasibility of AML MRD evaluation using spectral flow cytometry-based assays is demonstrated. Additionally, the ability of high-dimensional data to improve characterization of myelomonocytic maturation in routine clinical flow cytometry assessment was highlighted, which can aid the pathologist in visualization of aberrant maturational trajectories.

Discussed below is a third example associated with the various features disclosed herein. Aspects of the example can be implemented using the system 100 of FIG. 7. In general, aspects of the present disclosure associated with analyzing cell populations using machine learning to place cells into different cell groups (e.g., classes, subclasses, sub-subclasses, clusters, metaclusters, etc.) can be used to generate the data utilized in this example.

Full Integration of Automated Cell Typing and Interpretation of Clinical Flow Cytometry Data with a Laboratory Informatic System.

Introduction: Flow cytometry is an essential methodology in the diagnosis and prognostication of leukemias and lymphomas. While flow cytometry data quality has improved with increasing performance of instrumentation and the availability of novel fluorophores, the analysis of this data is complex, requiring significant training and time. Here, presented is a detailed experience with the clinical validation and use of a machine learning based flow cytometry analysis paired with a hematopathologist trained decision support system. This automated analysis program was further developed for end-to-end automated analysis from cytometer to laboratory informatic system (LIS) resulting.

Methods: Custom machine learning models were developed for the analysis of clinical flow cytometry data, acquired on cytometers with 31 markers and 29 fluorophores. These machines learning models were incorporated into a hematopathologist-trained clinical decision support system. This program was modified to run with continuous ingestion of FCS file output from the cytometers. Automated results and interpretations are automatically parsed and ingested by the LIS for resulting by the responsible hematopathologist.

Results: Machine learning based cell identification resulted in >99% accuracy in the identification of cell types at clinically relevant annotation levels. The combination of machine learning based cell typing and the clinical decision support system had a 100% sensitivity and 93% specificity for the detection of monotypic B-cell populations at a 0.1% threshold on traditional flow cytometers, which was further improved using spectral flow cytometry. The full automation of clinical flow cytometry data saved an estimated 40 hours per week in technician and pathologist time, with a volume of ˜400-500 cases per month.

Conclusions: Presented is a detailed pipeline for the integration of machine learning based cell typing and a clinical decision support system for flow cytometry data analysis. This method has been rigorously validated and implemented on both conventional and spectral platforms. The integration with an LIS further improves efficiency and mitigates errors while significantly improving turnaround time.

Discussed below is a fourth example associated with the various features disclosed herein. Aspects of the example can be implemented using the system 100 of FIG. 7. In general, aspects of the present disclosure associated with analyzing cell populations using machine learning to place cells into different cell groups (e.g., classes, subclasses, sub-subclasses, clusters, metaclusters, etc.) can be used to generate the data utilized in this example.

Development of a Machine Learning Screening Model Analyzing Different from Normal Maturation Patterns for MRD Testing on a Spectral Flow Cytometer

Introduction: Measurable residual disease (MRD) testing offers valuable prognostic information for patient outcomes in acute leukemia. Challenges in interpretation, however, result in high interobserver variance in identifying the abnormal leukemic immunophenotype at an analytical sensitivity of <0.01%, particularly in diseases that lack a leukemia-associated immunophenotype (LAIP). This is further compounded by the impact of different therapeutic regimens and clonal evolution altering the leukemic immunophenotypes. MRD can also be detected utilizing the different-from-normal (DfN) approach to identify deviations from normal maturational patterns. This method poses its own set of challenges as it requires expert knowledge of both normal and abnormal hematopoiesis, including transient or reactive abnormal hematopoiesis patterns that do not indicate leukemia. To meet these challenges, a highly supervised, machine learning-based DfN method to screen for MRD in acute myeloid leukemia and B-lymphoblastic leukemia was developed.

Methods: AML samples were analyzed using a clinically validated 31-color spectral flow cytometry panel. B-ALL samples were analyzed on a 21-color spectral panel. Both panels included a viability dye. All data were acquired on the Cytek Northern Lights System. Deep learning models were trained on normal bone marrow specimens. Maturational patterns were established using high-dimensional clustering results and previously-defined maturation patterns of myelomonocytic and lymphoid maturation. A hematopathologist-informed abnormality scoring system for DfN was developed and integrated with the models. A reporting system was developed to highlight 1) the abnormality scores at each stage of maturation and 2) the most likely abnormal populations for each sample.

Results: MRD identification by the model in both AML and B-ALL samples was verified by comparison with the immunophenotypes established at the time of clinical testing. Molecular MRD results, when available, were also used to verify the results. The model demonstrated sensitivity of ˜0.01% for a variety AML immunophenotypes, including those lacking typical LAIPs, and a sensitivity of up to ˜0.001% for B-ALL.

Conclusion: Emerging machine learning and artificial intelligence methods offer promising solutions for automating flow cytometry analysis. This screening model is a novel diagnostic aid that demonstrates high analytical sensitivity and effectively incorporates detailed hematopoietic maturational patterns. It is a valuable tool that aids a hematopathologist by providing comprehensive explanations of identified abnormal cell populations with MIRD results. While many artificial intelligence analysis methods serve to follow singular abnormal immunophenotypes per patient or are “black-boxed” and do not provide explanations, this program was designed to demonstrate all suggested abnormalities for final interpretation and does not require a diagnostic immunophenotype. These programs are currently undergoing clinical validation for future integration into routine clinical practice.

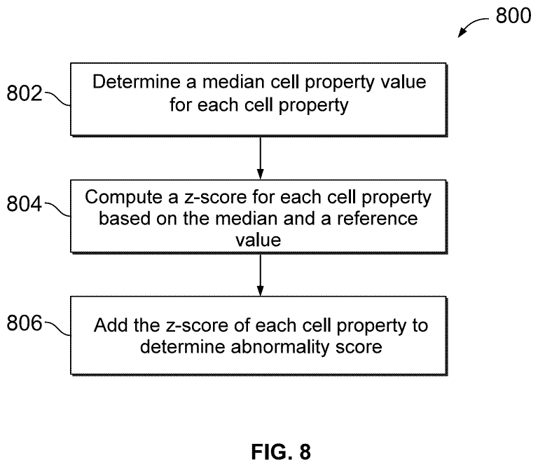

FIG. 8 illustrates a flowchart of a method 800 for determining an abnormality score for different cell groups within a plurality of cells. Method 800 can be used with any population of cells that has been sorted into a plurality of classes, including any of the examples discussed herein. For example, method 800 can be used to determine an abnormality score for any of the classes, subclasses, sub-subclasses, or any other grouping of cells discussed herein. By performing hierarchical classification as discussed herein, method 800 can be used to determined abnormality scores on accurate and granular cell classifications, which improves the specificity of the abnormality scoring in analyzing cells of a patient.

Method 800 illustrates the steps for determining the abnormality score for each respective cell group. Method 800 can be used repeatedly to determine the abnormality score all of the plurality of cells groups within the population of cells. Step 802 of method 800 includes determining a median value for each of a plurality of cell properties across all the cells in the respective cell group. These cell properties may include any suitable cell property. In some cases, the cell properties used to determine the abnormality score in method 800 are included in the one or more measurable parameters of the cells that are used by the machine learning model to place the cells into different classes/subclasses as described herein. For example, in some cases, the cell properties used in method 800 include properties that have a value associated with the presence and/or amount of a biomarker in the cells, such as the intensity and/or color of fluorescent emission from the cells that can be used to place the cells into different classes/subclasses.

Step 804 of method 800 includes, for each respective cell property, determining a z-score that is indicative of the difference between (i) the median value of the cell property for all the cells in the cell group and (ii) a reference value of the cell property for a reference cell group. The reference cell group includes a group of reference cells that have the same lineage as the cells in the respective cell group (e.g., are in the same class/subclass/sub-subclass), and have been determined to have a normal value for the cell property (e.g., the cells are from health and/or disease-free marrow/blood cohorts, and may be stratified by different characteristics such as age, instrument, site, etc.). This controls for expected maturation-stage shifts in the cells. In some implementations, the reference value of the cell property for the reference cell group is the mean value of the cell property for the reference cell group.

In some implementations, the z-score is determined by determining the absolute value of the difference between (i) the median value of the cell property for the cell group and (ii) the mean value of the cell property for the reference group, and dividing this value by the standard deviation of the value of the cell property for the reference cell group. Thus, the z-score in these implementations indicates the standard deviation of the value of the cell property in the cell group normalized to the standard deviation of the value of the cell property in the reference group. Determining the deviation from the reference group based on the cell class medians improves stability for rare cell populations and reduces noise from single-cell stochasticity.

In some implementations, a gating function can be applied to the z-score for each cell property. This gating function acts to indicate how many standard deviations away from the mean the value of the cell property for the cell group can be while still being considered to be normal. For example, the gating function can operate to compare the z-score to a predetermined threshold (e.g., an acceptable number of standard deviations), and if the z-score is greater than/greater than or equal to the threshold, the z-score remains the same. However, if the z-score is less than/less than or equal to the threshold, then the z-score for that cell property is set to 0. In some implementations, the threshold is an integer value, such as 3. The gating function acts to filter out benign variability and batch effects before the abnormality scores are determined.

In some implementations, step 804 includes applying a weighting function to the z-score of each cell property to generate a weighted z-score based on the importance of the respective cell property to the machine learning model in placing the cells in the different classes/subclasses/sub-subclasses. Cell properties that are more important to the placement of the cells (which may be based on internal weights of the machine learning model learned during training) are weighted heavier. In some implementations, the weighting function applies a decimal value between 0 and 1 the z-score of each cell property. The weighting function acts to emphasize cell aberrations that are known to be diagnostically relevant.

Step 806 of method 800 includes adding together the z-score of each respective cell property to determine the abnormality score for the cell group. In implementations where the weighting function is applied to the z-scores, step 806 includes adding together the weighted z-score of each respective cell property. In implementations where the gating function is applied, step 806 includes adding together the z-scores that are non-zero after the gating function has been applied. In implementations where both the weighting function and the gating function are applied, step 806 includes adding together the weighted z-scores that are non-zero after the gating function has been applied.

Method 800 can be repeatedly applied to different cell classes that are generated from flow cytometry data. In some implementations, additional steps can be taken to prepare the flow cytometry data for method 800. For example, the flow cytometry data can be analyzed and a compensation matrix applied to the individual events (cell detections). The detector channels in the flow cytometry data can be renamed to the specific cell properties they represent (although linear channels such as forward scatter angle and side scatter angle may be retained or renamed to “_lin” or the like. Quality filters can be applied to the flow cytometry data, such as an amine-reactive dye threshold for viability gating, or excluding low CD45 amounts as representing debris or non-leukocytes.

After the cells flow cytometry data is analyzed according to the methods disclosed herein to sort them into various cell groups (e.g., classes, subclasses, sub-subclasses, clusters, metaclusters, etc.), each event can be assigned a label indicating which class it belongs to. The per-class median value of the cell properties can then be determined by looking at, for each respective cell property and respective cell class, the value of the respective cell property for any event labeled as belonging to the respective cell class. Once the abnormality score is determined for each cell property, all of the abnormality scores for a given cell class are added together, and every event labeled as belong to the cell class can be assigned the abnormality score for that class.

Due to the use of cell group medians and/or the gating function, method 800 is robust to batch and sampling noise. The use of importance weighting and lineage-matching in the reference cell groups allows method 800 to be sensitive to clinically relevant deviations. Method 800 is also scalable to high-throughput MRD screening with automated narratives and figures.

Various outputs can be generated after the determination of the abnormality scores. For example, tables can be generated for each class showing the class label, the abnormality scores for the class, the counts of cells in the class, lineage labels for the class, etc. A per-cell CSV file can be generated with propagated scores for downstream gating and graphics. Various plots can be generated including standard orientation plots (e.g., the abnormality of CD33 cells vs. the abnormality of CD13 cells, the abnormality of HLA-DR cells vs. the abnormality of CD33 cells, etc.), abnormality-colored progenitor panels (e.g., CD34 axes), and LAIP (leukemia-associated immunophenotype) visualizations (e.g., CD34 vs. CD56/CD5/CD7/CD2). Narrative summaries can also be generated, including automated paragraph estimates (e.g., lineage fractions, viability, immature fractions, etc.) to support interpretation. The group-level abnormality scoring back-propagated to cells enables seamless integration with standard clinical workflows (including gating, density estimates, LAIP highlighting, etc.

One or more elements or aspects or steps, or any portion(s) thereof, from one or more of any of claims or Alternative Implementations below can be combined with one or more elements or aspects or steps, or any portion(s) thereof, from one or more of any of the other claims or Alternative Implementations or combinations thereof, to form one or more additional implementations and/or claims of the present disclosure.

### Alternative Implementations

Alternative Implementation 1. A method for classifying one or more cells, the method comprising: receiving data associated with the one or more cells, the data including, for each respective cell, information associated with one or more measurable parameters of the respective cell; inputting at least a portion of the data into a machine learning model; and receiving, from the machine learning model, an indication of an identity of at least one of the one or more cells.

Alternative Implementation 2. The method of Alternative Implementation 1, where the data associated with the one or more cells includes flow cytometry data.

Alternative Implementation 3. The method of Alternative Implementation 2, wherein the one or more measurable parameters of the respective cell include one or more parameters associated with scattering of light caused by the respective cell, one or more parameters associated with a biomarker of the respective cell, or both.

Alternative Implementation 4. The method of Alternative Implementation 3, wherein the one or more parameters associated with scattering of light caused by the respective cell include a forward scatter amount, a side scatter amount, a forward scatter time-of-flight, or any combination thereof.

Alternative Implementation 5. The method of Alternative Implementation 4, wherein the forward scatter amount includes a forward scatter area, a forward scatter angle, or both.

Alternative Implementation 6. The method of Alternative Implementation 4 or Alternative Implementation 5, wherein the side scatter amount includes a side scatter area, a side scatter angle, or both.

Alternative Implementation 7. The method of any one of Alternative Implementations 4 to 6, wherein the one or more parameters associated with the biomarker of the respective cell includes a presence of a predetermined molecule in the respective cell, an amount of the predetermined molecule in the respective cell, or both.

Alternative Implementation 8. The method of Alternative Implementation 7, wherein the one or more parameters associated with the biomarker of the respective cell includes an intensity of fluorescent emission from the respective cell, a color of fluorescent emission from the respective cell, or both.

Alternative Implementation 9. The method of Alternative Implementation 7 or Alternative Implementation 8, wherein the predetermined molecule includes a CD2 molecule, a CD3 molecule, a CD4 molecule, CD5 molecule, a CD7 molecule, a CD8 molecule, a CD10 molecule, a CD11 molecule, a CD13 molecule, a CD14 molecule, a CD16 molecule, a CD19 molecule, a CD20 molecule, a CD22 molecule, a CD23 molecule, a CD33 molecule, a CD34 molecule, a CD45 molecule, a CD64 molecule, a CD117 molecule, an HLA-DR molecule, or any combination thereof.

Alternative Implementation 10. The method of any one of Alternative Implementations 7 to 9, wherein the predetermined molecule includes a cluster of differentiation molecule, an antigen, an antibody, an immunoglobulin chain, or any combination thereof.

Alternative Implementation 11. The method of Alternative Implementation 10, wherein the immunoglobulin chain includes a kappa (κ) light chain, a lambda (λ) light chain, a gamma (γ) heavy chain, a delta (δ) heavy chain, an alpha (α) heavy chain, a mu (μ) heavy chain, an epsilon (ϵ) heavy chain, or any combination thereof.

Alternative Implementation 12. The method of any one of Alternative Implementations 1 to 11, wherein the indication of the identity of each respective cell includes a placement of the respective cell within each of a plurality of classes based on the one or more measurable parameters, each respective class being associated with a plurality of potential combinations of one or more cell characteristics.

Alternative Implementation 13. The method of Alternative Implementation 12, wherein each of the plurality of classes includes a plurality of subclasses, each respective subclass of each respective class being associated with a distinct one of the plurality of potential combinations of cell characteristics of the respective class.

Alternative Implementation 14. The method of Alternative Implementation 13, wherein placement of each respective cell within a respective class of the plurality of classes includes a selection of one of the plurality of subclasses of the respective class.

Alternative Implementation 15. The method of any one of Alternative Implementations 12 to 14, wherein the plurality of distinct classes are determined by one or more predetermined clustering algorithms.

Alternative Implementation 16. The method of Alternative Implementation 15, wherein the one or more predetermined clustering algorithms includes FlowSOM, Phenograph, or both.

Alternative Implementation 17. The method of any one of Alternative Implementations 12 to 16, wherein the plurality of classes includes a cluster class, and wherein the plurality of subclasses of the cluster class include a plurality of distinct clusters.

Alternative Implementation 18. The method of any one of Alternative Implementations 12 to 17, wherein the plurality of classes includes a immunophenotype class, and wherein each of the plurality of subclasses of the immunophenotype class is associated with a distinct combination of (i) a presence of one or more cluster of differentiation (CD) molecules, one or more cell surface markers, one or more intracellular markers, or any combination thereof; (ii) a diminished presence of the one or more CD molecules, the one or more cell surface markers, the one or more intracellular markers, or any combination thereof; (iii) an absence of the one or more CD molecules, the one or more cell surface markers, the one or more intracellular markers, or any combination thereof; or (iv) any combination of (i)-(iii).

Alternative Implementation 19. The method of Alternative Implementation 18, wherein the plurality of subclasses of the immunophenotype class includes: (i) a first subclass associated with the presence of a CD10 molecule; (ii) a second subclass associated with the presence of a CD5 molecule, the diminished presence of a CD20 molecule, the diminished presence of a CD22 molecule, and the absence of a CD23 molecule; (iii) a third subclass associated with the presence of the CD5 molecule, the presence of the CD20 molecule, the presence of the CD22 molecule, and the presence of the CD23 molecule; (iv) a fourth subclass associated with the presence of the CD5 molecule, the diminished presence of the CD20 molecule, the diminished presence of the CD22 molecule, and the presence of the CD23 molecule; (v) a fifth subclass associated with the diminished presence of CD5 molecule, the diminished presence of the CD20 molecule, the diminished presence of the CD22 molecule, and the absence of the CD23 molecule; (vi) a sixth subclass associated with the diminished presence with the CD5 molecule, the diminished presence of the CD20 molecule, the diminished presence of the CD22 molecule, and the diminished presence of the CD23 molecule; (vii) a seventh subclass associated with the absence of the CD5 molecule, the presence of the CD20 molecule, the diminished presence of the CD22 molecule, and the absence of the CD23 molecule; (viii) an eighth subclass associated with the absence of the CD5 molecule, the presence of the CD20 molecule, the diminished presence of the CD22 molecule, and the diminished presence of the CD23 molecule; (ix) a ninth subclass associated with the absence of the CD5 molecule, the presence of the CD20 molecule, the diminished presence of the CD22 molecule, and the diminished presence of the CD23 molecule; and (x) a tenth subclass associated with the absence of the CD5 molecule, the presence of the CD20 molecule, the presence of the CD22 molecule, and the absence of the CD23 molecule.

Alternative Implementation 20. The method of any one of Alternative Implementations 12 to 19, wherein the plurality of classes includes a CD5/CD10 class, and wherein the plurality of subclasses of the CD5/CD10 class includes a first subclass associated with a presence of a CD10 molecule, a second subclass associated with a presence of a CD5 molecule, a third subclass associated with a diminished presence of the CD5 molecule, and a fourth subclass associated with an absence of the CD5 molecule.

Alternative Implementation 21. The method of any one of Alternative Implementations 12 to 20, wherein the plurality of classes includes a B-cell normality class, and wherein the plurality of subclasses of the B-cell normality class includes a normal B-cell subclass and an abnormal B-cell subclass.

Alternative Implementation 22. The method of any one of Alternative Implementations 1 to 21, wherein the indication of the identity of each respective cell includes an indication of whether the respective cell is a B-cell.

Alternative Implementation 23. The method of any one of Alternative Implementations 1 to 22, wherein the indication of the identity of each respective cell includes an indication of whether the respective cell is a normal B-cell or an abnormal B-cell.

Alternative Implementation 24. The method of any one of Alternative Implementations 1 to 23, wherein the indication of the identity of each respective cell includes an indication of a predefined phenotype of a plurality of predefined phenotypes that the respective cell belongs to.

Alternative Implementation 25. The method of any one of Alternative Implementations 1 to 24, wherein the indication of the identity of each respective cell includes an indication of an immunophenotype of the respective cell.

Alternative Implementation 26. The method of any one of Alternative Implementations 1 to 25, wherein the indication of the identity of each respective cell includes a placement of each respective cell into one of a plurality of cell classes that include B-cells, normal B-cells, abnormal B-cells, B-cells having any combination of a presence or an absence of one or more cluster of differentiation (CD) molecules, T-cells, normal T-cells, abnormal T-cells, T-cells having any combination of a presence or an absence of one or more cluster of differentiation (CD) molecules, double-negative T-cells, cells negative for a CD45 molecule, granulocytes, monocytes, monocytes with a diminished presence of a CD4 molecule, monocytes with a presence of a CD56 molecule, mature cells, immature cells, natural killer (NK) cells, NK cells with an absence of a CD2 molecule and a CD5 molecule, NK cells with an absence of the CD5 molecule, plasma cells, B-lymphoblasts, T-lymphoblasts, or any combination thereof.

Alternative Implementation 27. The method of any one of Alternative Implementations 1 to 26, wherein the one or more cells includes a plurality of cells, and the data inputted into the machine learning model includes data associated with each of the plurality of cells, the method further comprising: receiving the indication of the identity of each of the plurality of cells, the indication of the identity of each of the plurality of cells including a value of a first parameter associated with a first biomarker of the cells and a value of a second parameter associated with a second biomarker of the cells; and identifying a plurality of distinct maturation stages of the plurality of cells based on the value of the first parameter and the value of the second parameter for each of the plurality of cells.

Alternative Implementation 28. The method of Alternative Implementation 27, wherein the plurality of cells are myeloid cells, the first biomarker is a CD34 molecule, and the second biomarker is a CD117 molecule.

Alternative Implementation 29. The method of Alternative Implementation 27, wherein the plurality of cells are myeloid cells, the first biomarker is a CD13 molecule, and the second biomarker is a CD15 molecule.

Alternative Implementation 30. The method of Alternative Implementation 27, wherein the plurality of cells are monocytes, the first biomarker is a CD64 molecule, and the second biomarker is a CD14 molecule.

Alternative Implementation 31. The method of any one of Alternative Implementations 1 to 30, wherein the machine learning model is trained to: sort each respective cell into one of a first plurality of cell classes, each of the first plurality of cell classes corresponding to a distinct combination of one or more cell characteristics; and sort each respective cell into one of a second plurality of cell classes, each of the second plurality of cell classes corresponding to a second distinct combination of the one or more cell characteristics.

Alternative Implementation 32. The method of Alternative Implementation 31, wherein a number of cell classes in the first plurality of cell classes is greater than a number of cell classes in the second plurality of cell classes.

Alternative Implementation 33. The method of any one of Alternative Implementations 1 to 32, wherein the trained machined learning model includes a random forest model having (i) a plurality of trees and (ii) a voting module.

Alternative Implementation 34. The method of Alternative Implementation 33, wherein each of the plurality of trees is configured to generate an independent indication of the identity of each respective cell.

Alternative Implementation 35. The method of Alternative Implementation 34, wherein each of the plurality of trees is configured to perform an independent placement of the respective cell within at least one of the plurality of classes.

Alternative Implementation 36. The method of Alternative Implementation 34 or Alternative Implementation 35, wherein the voting module is configured to select the independent indication of the identity of the respective cell of one of the plurality of trees.

Alternative Implementation 37. The method of any one of Alternative Implementations 34 to 36, wherein the voting module is configured to determine a weighted average of the independent indication of the identity of the cell performed by the plurality of trees.

Alternative Implementation 38. The method of any one of Alternative Implementations 1 to 37, wherein the machine learning model includes a k-nearest neighbor model with k=7.

Alternative Implementation 39. The method of any one of Alternative Implementations 1 to 38, wherein the machine learning model includes a neural network, a k-nearest neighbor algorithm, a decision tree, a random forest model, or any combination thereof.

Alternative Implementation 40. The method of any one of Alternative Implementations 1 to 39, wherein the machine learning model is trained using a training data set, the training data set including (i) raw flow cytometry data associated with a plurality a cells, and (ii) for each respective cell of the plurality of cells, a determination of the identity of the respective cell.

Alternative Implementation 41. The method of any one of Alternative Implementations 1 to 40, further comprising: analyzing the indication of the identity of each of the one or more cells; and based at least in part on the analysis, generating a graphical representation of the identity of at least one of the one or more cells.

Alternative Implementation 42. The method of any one of Alternative Implementations 1 to 41, further comprising: analyzing the indication of the identity of each of the one or more cells; and based at least in part on the analysis, generating a text description of the identity of at least one of the one or more cells.

Alternative Implementation 43. The method of any one of Alternative Implementations 1 to 42, wherein the one or more cells were obtained from an individual, and wherein the method further comprises: analyzing the indication of the identity of each of the one or more cells; and based at least in part on the analysis, generating a recommendation for one or more clinical tests for the individual to undergo.

Alternative Implementation 44. The method of any one of Alternative Implementations 1 to 43, wherein the one or more cells were obtained from an individual, and wherein the method further comprises: analyzing the indication of the identity of each of the one or more cells; and based at least in part on the analysis, generating a diagnosis for the individual.

Alternative Implementation 45. The method of any one of Alternative Implementations 1 to 44, wherein the one or more cells were obtained from an individual, and wherein the method further comprises: analyzing the indication of the identity of each of the one or more cells; and based at least in part on the analysis, generating a template for reporting the identity of each of the one or more cells.

Alternative Implementation 46. The method of any one of Alternative Implementations 1 to 45, wherein the one or more cells were obtained from an individual, and wherein the method further comprises: analyzing the indication of the identity of each of the one or more cells; and displaying, on a display device, (i) a diagnosis for the individual based at least in part on the analysis, (ii) one or more graphical representations of the one or more cells, (iii) or (iii) both (i) and (ii).

Alternative Implementation 47. The method of any one of Alternative Implementations 12 to 46, further comprising generating an abnormality score for each of a plurality of cell groups, the plurality of cell groups including (i) the plurality of classes, (ii) the plurality of subclasses of each of the plurality of classes, (iii) a plurality of sub-subclasses of each of the plurality of subclasses of each of the plurality of classes, (iv) any plurality of groups that the machine learning model places the plurality of cells into, or (v) any combination of (i)-(iv).

Alternative Implementation 48. The method of Alternative Implementation 47, wherein generating the abnormality score for each respective cell group of the plurality of cell groups includes: determining a median value of each of a plurality of cell properties across the cells in the respective cell group; for each respective cell property, determine a z-score indicative of a difference between (i) the median value of the respective cell property for the respective cell group and (ii) a reference value of the respective cell property for a reference cell group; and add the z-score of each respective cell property to determine the abnormality score for the respective cell group.

Alternative Implementation 49. The method of Alternative Implementation 48, wherein the reference value of the respective cell property for the reference cell group is a mean value of the respective cell property for the reference cell group.

Alternative Implementation 50. The method of Alternative Implementation 49, wherein determining the z-score for each respective cell property includes: determining an absolute value of the difference between (i) the median value of the respective cell property for the respective cell group and (ii) the mean value of the respective cell property for the reference cell group; and dividing the absolute value of the difference by a standard deviation of the respective cell property for the reference cell group.

Alternative Implementation 51. The method of any one of Alternative Implementations 48 to 50, further comprising, for each respective cell group, applying a gating function to the z-score for each respective cell property such that (i) the value of the z-score remains unchanged if the value of the z-score is greater than a predetermined threshold and (ii) the value of the z-score is set to 0 if the value of the z-score is less than or equal to the predetermined threshold.

Alternative Implementation 52. The method of Alternative Implementation 51, wherein the predetermined threshold is an integer value.

Alternative Implementation 53. The method of Alternative Implementation 51 or Alternative Implementation 52, wherein the predetermined threshold is 3.

Alternative Implementation 54. The method of any one of Alternative Implementations 50 to 53, wherein the reference cell group for each respective cell group includes reference cells having an identical lineage as the cells in the respective cell group.

Alternative Implementation 55. The method of any one of Alternative Implementations 48 to 54, wherein the one or more measurable parameters used by the machine learning model to place each respective cell into one of the plurality of cell groups includes the plurality of cell properties.

Alternative Implementation 56. The method of Alternative Implementation 55, further comprising, for each respective cell group, applying a weighting function to the z-score of each respective cell property to generate a weighted z-score for each respective cell group based on an importance of the respective cell property in placing each respective cell into one of the plurality of cell groups, and wherein the abnormality score for each respective cell group is determined by adding the weighted z-score of each respective cell property for the respective cell group.

Alternative Implementation 57. The method of Alternative Implementation 55 or Alternative Implementation 56, wherein the plurality of cell properties include a plurality of properties having a value associated with a presence and/or an amount of a biomarker in the respective cell.

Alternative Implementation 58. A system for classifying one or more cells comprising: a memory device having stored thereon machine-readable instructions; and a control system including one or more processors configured to execute the machine-readable instructions to implement the method of any one of Alternative Implementations 1 to 57.

Alternative Implementation 59. A system for classifying one or more cells, the system comprising a control system configured to implement the method of any one of Alternative Implementations 1 to 57.

Alternative Implementation 60. A computer program product comprising instructions which, when executed by a computer, cause the computer to carry out the method of any one of Alternative Implementations 1 to 57.

Alternative Implementation 61. The computer program product of Alternative Implementation 60, wherein the computer program product is a non-transitory computer readable medium.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A method for classifying one or more cells, the method comprising:
receiving data associated with the one or more cells, the data including, for each respective cell, information associated with one or more measurable parameters of the respective cell;
inputting at least a portion of the data into a machine learning model; and
receiving, from the machine learning model, an indication of an identity of at least one of the one or more cells.

2. The method of claim 1, where the data associated with the one or more cells includes flow cytometry data.

3. The method of claim 2, wherein the one or more measurable parameters of the respective cell include one or more parameters associated with scattering of light caused by the respective cell, one or more parameters associated with a biomarker of the respective cell, or both.

4. The method of claim 3, wherein the one or more parameters associated with scattering of light caused by the respective cell include a forward scatter amount, a side scatter amount, a forward scatter time-of-flight, or any combination thereof.

5. The method of claim 4, wherein the forward scatter amount includes a forward scatter area, a forward scatter angle, or both.

6. The method of claim 4 or claim 5, wherein the side scatter amount includes a side scatter area, a side scatter angle, or both.

7. The method of any one of claims 4 to 6, wherein the one or more parameters associated with the biomarker of the respective cell includes a presence of a predetermined molecule in the respective cell, an amount of the predetermined molecule in the respective cell, or both.

8. The method of claim 7, wherein the one or more parameters associated with the biomarker of the respective cell includes an intensity of fluorescent emission from the respective cell, a color of fluorescent emission from the respective cell, or both.

9. The method of claim 7 or claim 8, wherein the predetermined molecule includes a CD2 molecule, a CD3 molecule, a CD4 molecule, CD5 molecule, a CD7 molecule, a CD8 molecule, a CD10 molecule, a CD11 molecule, a CD13 molecule, a CD14 molecule, a CD16 molecule, a CD19 molecule, a CD20 molecule, a CD22 molecule, a CD23 molecule, a CD33 molecule, a CD34 molecule, a CD45 molecule, a CD64 molecule, a CD117 molecule, an HLA-DR molecule, or any combination thereof.

10. The method of any one of claims 7 to 9, wherein the predetermined molecule includes a cluster of differentiation molecule, an antigen, an antibody, an immunoglobulin chain, or any combination thereof.

11. The method of claim 10, wherein the immunoglobulin chain includes a kappa (κ) light chain, a lambda (λ) light chain, a gamma (γ) heavy chain, a delta (δ) heavy chain, an alpha (α) heavy chain, a mu (μ) heavy chain, an epsilon (ϵ) heavy chain, or any combination thereof.

12. The method of any one of claims 1 to 11, wherein the indication of the identity of each respective cell includes a placement of the respective cell within each of a plurality of classes based on the one or more measurable parameters, each respective class being associated with a plurality of potential combinations of one or more cell characteristics.

13. The method of claim 12, wherein each of the plurality of classes includes a plurality of subclasses, each respective subclass of each respective class being associated with a distinct one of the plurality of potential combinations of cell characteristics of the respective class.

14. The method of claim 13, wherein placement of each respective cell within a respective class of the plurality of classes includes a selection of one of the plurality of subclasses of the respective class.

15. The method of any one of claims 12 to 14, wherein the plurality of distinct classes are determined by one or more predetermined clustering algorithms.

16. The method of claim 15, wherein the one or more predetermined clustering algorithms includes FlowSOM, Phenograph, or both.

17. The method of any one of claims 12 to 16, wherein the plurality of classes includes a cluster class, and wherein the plurality of subclasses of the cluster class include a plurality of distinct clusters.

18. The method of any one of claims 12 to 17, wherein the plurality of classes includes a immunophenotype class, and wherein each of the plurality of subclasses of the immunophenotype class is associated with a distinct combination of (i) a presence of one or more cluster of differentiation (CD) molecules, one or more cell surface markers, one or more intracellular markers, or any combination thereof; (ii) a diminished presence of the one or more CD molecules, the one or more cell surface markers, the one or more intracellular markers, or any combination thereof; (iii) an absence of the one or more CD molecules, the one or more cell surface markers, the one or more intracellular markers, or any combination thereof; or (iv) any combination of (i)-(iii).

19. The method of claim 18, wherein the plurality of subclasses of the immunophenotype class includes:
(i) a first subclass associated with the presence of a CD10 molecule;
(ii) a second subclass associated with the presence of a CD5 molecule, the diminished presence of a CD20 molecule, the diminished presence of a CD22 molecule, and the absence of a CD23 molecule;
(iii) a third subclass associated with the presence of the CD5 molecule, the presence of the CD20 molecule, the presence of the CD22 molecule, and the presence of the CD23 molecule;
(iv) a fourth subclass associated with the presence of the CD5 molecule, the diminished presence of the CD20 molecule, the diminished presence of the CD22 molecule, and the presence of the CD23 molecule;
(v) a fifth subclass associated with the diminished presence of CD5 molecule, the diminished presence of the CD20 molecule, the diminished presence of the CD22 molecule, and the absence of the CD23 molecule;
(vi) a sixth subclass associated with the diminished presence with the CD5 molecule, the diminished presence of the CD20 molecule, the diminished presence of the CD22 molecule, and the diminished presence of the CD23 molecule;
(vii) a seventh subclass associated with the absence of the CD5 molecule, the presence of the CD20 molecule, the diminished presence of the CD22 molecule, and the absence of the CD23 molecule;
(viii) an eighth subclass associated with the absence of the CD5 molecule, the presence of the CD20 molecule, the diminished presence of the CD22 molecule, and the diminished presence of the CD23 molecule;
(ix) a ninth subclass associated with the absence of the CD5 molecule, the presence of the CD20 molecule, the diminished presence of the CD22 molecule, and the diminished presence of the CD23 molecule; and
(x) a tenth subclass associated with the absence of the CD5 molecule, the presence of the CD20 molecule, the presence of the CD22 molecule, and the absence of the CD23 molecule.

20. The method of any one of claims 12 to 19, wherein the plurality of classes includes a CD5/CD10 class, and wherein the plurality of subclasses of the CD5/CD10 class includes a first subclass associated with a presence of a CD10 molecule, a second subclass associated with a presence of a CD5 molecule, a third subclass associated with a diminished presence of the CD5 molecule, and a fourth subclass associated with an absence of the CD5 molecule.

21. The method of any one of claims 12 to 20, wherein the plurality of classes includes a B-cell normality class, and wherein the plurality of subclasses of the B-cell normality class includes a normal B-cell subclass and an abnormal B-cell subclass.

22. The method of any one of claims 1 to 21, wherein the indication of the identity of each respective cell includes an indication of whether the respective cell is a B-cell.

23. The method of any one of claims 1 to 22, wherein the indication of the identity of each respective cell includes an indication of whether the respective cell is a normal B-cell or an abnormal B-cell.

24. The method of any one of claims 1 to 23, wherein the indication of the identity of each respective cell includes an indication of a predefined phenotype of a plurality of predefined phenotypes that the respective cell belongs to.

25. The method of any one of claims 1 to 24, wherein the indication of the identity of each respective cell includes an indication of an immunophenotype of the respective cell.

26. The method of any one of claims 1 to 25, wherein the indication of the identity of each respective cell includes a placement of each respective cell into one of a plurality of cell classes that include B-cells, normal B-cells, abnormal B-cells, B-cells having any combination of a presence or an absence of one or more cluster of differentiation (CD) molecules, T-cells, normal T-cells, abnormal T-cells, T-cells having any combination of a presence or an absence of one or more cluster of differentiation (CD) molecules, double-negative T-cells, cells negative for a CD45 molecule, granulocytes, monocytes, monocytes with a diminished presence of a CD4 molecule, monocytes with a presence of a CD56 molecule, mature cells, immature cells, natural killer (NK) cells, NK cells with an absence of a CD2 molecule and a CD5 molecule, NK cells with an absence of the CD5 molecule, plasma cells, B-lymphoblasts, T-lymphoblasts, or any combination thereof.

27. The method of any one of claims 1 to 26, wherein the one or more cells includes a plurality of cells, and the data inputted into the machine learning model includes data associated with each of the plurality of cells, the method further comprising:
receiving the indication of the identity of each of the plurality of cells, the indication of the identity of each of the plurality of cells including a value of a first parameter associated with a first biomarker of the cells and a value of a second parameter associated with a second biomarker of the cells; and
identifying a plurality of distinct maturation stages of the plurality of cells based on the value of the first parameter and the value of the second parameter for each of the plurality of cells.

28. The method of claim 27, wherein the plurality of cells are myeloid cells, the first biomarker is a CD34 molecule, and the second biomarker is a CD117 molecule.

29. The method of claim 27, wherein the plurality of cells are myeloid cells, the first biomarker is a CD13 molecule, and the second biomarker is a CD15 molecule.

30. The method of claim 27, wherein the plurality of cells are monocytes, the first biomarker is a CD64 molecule, and the second biomarker is a CD14 molecule.

31. The method of any one of claims 1 to 30, wherein the machine learning model is trained to:
sort each respective cell into one of a first plurality of cell classes, each of the first plurality of cell classes corresponding to a distinct combination of one or more cell characteristics; and
sort each respective cell into one of a second plurality of cell classes, each of the second plurality of cell classes corresponding to a second distinct combination of the one or more cell characteristics.

32. The method of claim 31, wherein a number of cell classes in the first plurality of cell classes is greater than a number of cell classes in the second plurality of cell classes.

33. The method of any one of claims 1 to 32, wherein the trained machined learning model includes a random forest model having (i) a plurality of trees and (ii) a voting module.

34. The method of claim 33, wherein each of the plurality of trees is configured to generate an independent indication of the identity of each respective cell.

35. The method of claim 34, wherein each of the plurality of trees is configured to perform an independent placement of the respective cell within at least one of the plurality of classes.

36. The method of claim 34 or claim 35, wherein the voting module is configured to select the independent indication of the identity of the respective cell of one of the plurality of trees.

37. The method of any one of claims 34 to 36, wherein the voting module is configured to determine a weighted average of the independent indication of the identity of the cell performed by the plurality of trees.

38. The method of any one of claims 1 to 37, wherein the machine learning model includes a k-nearest neighbor model with k=7.

39. The method of any one of claims 1 to 38, wherein the machine learning model includes a neural network, a k-nearest neighbor algorithm, a decision tree, a random forest model, or any combination thereof.

40. The method of any one of claims 1 to 39, wherein the machine learning model is trained using a training data set, the training data set including (i) raw flow cytometry data associated with a plurality a cells, and (ii) for each respective cell of the plurality of cells, a determination of the identity of the respective cell.

41. The method of any one of claims 1 to 40, further comprising:
analyzing the indication of the identity of each of the one or more cells; and
based at least in part on the analysis, generating a graphical representation of the identity of at least one of the one or more cells.

42. The method of any one of claims 1 to 41, further comprising:
analyzing the indication of the identity of each of the one or more cells; and
based at least in part on the analysis, generating a text description of the identity of at least one of the one or more cells.

43. The method of any one of claims 1 to 42, wherein the one or more cells were obtained from an individual, and wherein the method further comprises:
analyzing the indication of the identity of each of the one or more cells; and
based at least in part on the analysis, generating a recommendation for one or more clinical tests for the individual to undergo.

44. The method of any one of claims 1 to 43, wherein the one or more cells were obtained from an individual, and wherein the method further comprises:
analyzing the indication of the identity of each of the one or more cells; and
based at least in part on the analysis, generating a diagnosis for the individual.

45. The method of any one of claims 1 to 44, wherein the one or more cells were obtained from an individual, and wherein the method further comprises:
analyzing the indication of the identity of each of the one or more cells; and
based at least in part on the analysis, generating a template for reporting the identity of each of the one or more cells.

46. The method of any one of claims 1 to 45, wherein the one or more cells were obtained from an individual, and wherein the method further comprises:
analyzing the indication of the identity of each of the one or more cells; and
displaying, on a display device, (i) a diagnosis for the individual based at least in part on the analysis, (ii) one or more graphical representations of the one or more cells, (iii) or (iii) both (i) and (ii).

47. The method of any one of claims 12 to 46, further comprising generating an abnormality score for each of a plurality of cell groups, the plurality of cell groups including (i) the plurality of classes, (ii) the plurality of subclasses of each of the plurality of classes, (iii) a plurality of sub-subclasses of each of the plurality of subclasses of each of the plurality of classes, (iv) any plurality of groups that the machine learning model places the plurality of cells into, or (v) any combination of (i)-(iv).

48. The method of claim 47, wherein generating the abnormality score for each respective cell group of the plurality of cell groups includes:
determining a median value of each of a plurality of cell properties across the cells in the respective cell group;
for each respective cell property, determine a z-score indicative of a difference between (i) the median value of the respective cell property for the respective cell group and (ii) a reference value of the respective cell property for a reference cell group; and
add the z-score of each respective cell property to determine the abnormality score for the respective cell group.

49. The method of claim 48, wherein the reference value of the respective cell property for the reference cell group is a mean value of the respective cell property for the reference cell group.

50. The method of claim 49, wherein determining the z-score for each respective cell property includes:
determining an absolute value of the difference between (i) the median value of the respective cell property for the respective cell group and (ii) the mean value of the respective cell property for the reference cell group; and
dividing the absolute value of the difference by a standard deviation of the respective cell property for the reference cell group.

51. The method of any one of claims 48 to 50, further comprising, for each respective cell group, applying a gating function to the z-score for each respective cell property such that (i) the value of the z-score remains unchanged if the value of the z-score is greater than a predetermined threshold and (ii) the value of the z-score is set to 0 if the value of the z-score is less than or equal to the predetermined threshold.

52. The method of claim 51, wherein the predetermined threshold is an integer value.

53. The method of claim 51 or claim 52, wherein the predetermined threshold is 3.

54. The method of any one of claims 50 to 53, wherein the reference cell group for each respective cell group includes reference cells having an identical lineage as the cells in the respective cell group.

55. The method of any one of claims 48 to 54, wherein the one or more measurable parameters used by the machine learning model to place each respective cell into one of the plurality of cell groups includes the plurality of cell properties.

56. The method of claim 55, further comprising, for each respective cell group, applying a weighting function to the z-score of each respective cell property to generate a weighted z-score for each respective cell group based on an importance of the respective cell property in placing each respective cell into one of the plurality of cell groups, and wherein the abnormality score for each respective cell group is determined by adding the weighted z-score of each respective cell property for the respective cell group.

57. The method of claim 55 or claim 56, wherein the plurality of cell properties include a plurality of properties having a value associated with a presence and/or an amount of a biomarker in the respective cell.

58. A system for classifying one or more cells comprising:
a memory device having stored thereon machine-readable instructions; and
a control system including one or more processors configured to execute the machine-readable instructions to implement the method of any one of claims 1 to 57.

59. A system for classifying one or more cells, the system comprising a control system configured to implement the method of any one of claims 1 to 57.

60. A computer program product comprising instructions which, when executed by a computer, cause the computer to carry out the method of any one of claims 1 to 57.

61. The computer program product of claim 60, wherein the computer program product is a non-transitory computer readable medium.
